(19) United States
(12) Patent Application Publication
(10) Pub. No.: US 2021/0142906 A1
(43) Pub. Date: May 13, 2021

(54) METHODS AND SYSTEMS FOR ORAL MICROBIOME ANALYSIS

(71) Applicant: Predentome, Inc., San Diego, CA (US)

(72) Inventor: Soheil BIGDELI, San Diego, CA (US)

(21) Appl. No.: 17/152,657

(22) Filed: Jan. 19, 2021

Related U.S. Application Data
(63) Continuation of application No. PCT/US2019/042678, filed on Jul. 19, 2019.
(60) Provisional application No. 62/701,232, filed on Jul. 20, 2018.

(51) Int. Cl. G16H 50/20; G16H 50/30; G16H 50/50

(52) U.S. Cl. CPC G16H 50/20; G16H 50/50; G16H 50/30

(57) ABSTRACT

Described are platforms, systems, and methods for providing a recommendation for an individual regarding oral health based on an analysis of an oral biological sample from the individual. In one aspect, a method includes receiving a plurality of sequence reads of an oral biological sample collected from an individual, wherein each of the sequence reads corresponding to one or more nucleic acid molecules from at least one food source or at least one microorganism; determining an abundance by: taxonomically classifying the sequence reads to identify the at least one food source or the at least one microorganism; and quantifying the one or more nucleic acid molecules; processing the abundance through a machine learning algorithm to determine an indicator of an oral or gum disease in the individual; and providing, to a user interface, a recommendation for the individual regarding oral health.

### CROSS-REFERENCE

This patent application in a continuation of International Patent Application No. PCT/US2019/042,678, filed Jul. 19, 2019, which claims the benefit of U.S. Provisional Patent Application No. 62/701,232, filed Jul. 20, 2018, which are incorporated herein by reference in their entirety.

### SUMMARY

Disclosed herein, in certain embodiments, are methods of predicting an oral and/or gum disease in an individual in need thereof, comprising: receiving a plurality of sequence reads from an oral biological sample of the individual, the plurality of sequence reads corresponding to one or more nucleic acid molecules from at least one food source; taxonomically classifying the plurality of sequence reads to identify the at least one food source; quantifying the one or more nucleic acid molecules from the identified at least one food source, thereby generating a calculated abundance of the identified at least one food source; and applying a machine learning algorithm, to: compare the calculated abundance to a cutoff abundance value associated with a healthy oral biological sample or with a diseased oral biological sample; calculate a correlation value between the calculated abundance and the cutoff abundance value; and provide an indicator of an oral and/or gum disease in the individual based on the correlation value, wherein the calculated abundance is used to continually retrain the machine learning algorithm to adjust the cutoff abundance value.

In some embodiments, the at least one food source is a plant source, an animal source, or an edible fungal source. In some embodiments, the plant source is a plant from the kingdom of Thallophyta, Pteridophyta, or Spermatophyta. In some embodiments, the animal source is an invertebrate animal source or a vertebrate animal source. In some embodiments, the edible fungal source is a fungus from the Ascomycota phylum, Basidiomycota phylum, Blastocladiomycota phylum, Chytridiomycota phylum, Glomeromycota phylum, Microsporidia phylum, or Neocallimastigomycota phylum. In some embodiments, the oral biological sample comprises one or more nucleic acid molecules from a microorganism. In some embodiments, the microorganism is a bacterium, a virus, or a fungus. In some embodiments, the method further comprises applying steps a), b), and c) to the one or more nucleic acid molecules from the microorganism. In some embodiments, the calculated abundance of the at least one food source and/or a calculated abundance of the microorganism are used to estimate a potential hydrogen (pH) value of the oral cavity of the individual.

In some embodiments, the machine learning algorithm further compares the calculated abundance of the at least one food source and/or the calculated abundance of the microorganism to the pH value. In some embodiments, the method further comprises receiving a survey datum from the individual, the survey datum being coincident with the oral biological sample. In some embodiments, the machine learning algorithm further converts the survey datum into a quantitative score. In some embodiments, the survey datum is used to continually retrain the machine learning algorithm to adjust the cutoff abundance value.

In some embodiments, the calculated abundance is associated with the survey datum. In some embodiments, the survey datum comprises dietary information, medical history, medical records, lifestyle information, or any combinations thereof, of the individual. In some embodiments, the lifestyle information comprises information regarding alcohol use, tobacco use, dental hygiene habits, or any combination thereof, of the individual. In some embodiments, the machine learning algorithm compares and correlates the calculated abundance to a gene expression profile of at least one gene in the oral biological sample. In some embodiments, the machine learning algorithm further compares the gene expression profile of the at least one gene in the oral biological sample to a reference gene expression profile of the at least one gene.

In some embodiments, the at least one gene is differentially expressed in individuals who have an oral and/or gum disease as compared to individuals who do not have an oral and/or gum disease. In some embodiments, the reference gene expression profile is an average expression profile of the at least one gene in an oral biological sample of individuals who do not have an oral and/or gum disease. In some embodiments, the at least one gene is selected from Matrix MetalloProtease-10 (MMP10), Matrix MetalloProtease-14 (MMP14), Matrix MetalloProtease-16 (MMP16), Metallophosphoesterase Domain Containing 2 (MPPED2), Actinin Alpha 2 (ACTN2), vitamin D receptor (VDR), FccRIIA, Interleukin1-alpha (IL1-alpha), and Interleukin1-beta (IL1-beta).

In some embodiments, the machine learning algorithm is a random forest model, a t-distributed stochastic neighbor embedding (tSNE) model, an artificial neural network model, a decision tree model, a k-nearest neighbor (kNN) model, a principal component analysis (PCA) model, a transfer component analysis (TCA) classifier, a deep neural network model, a support vector machine model, or a linear classification model. In some embodiments, the machine learning algorithm recognizes an abundance pattern in the calculated abundance. In some embodiments, the oral biological sample is a saliva sample, a biofilm sample, a dental tissue sample, a dental plaque sample, a tartar sample, a dental calculus sample, or any combination thereof. In some embodiments, the oral biological sample is collected from: a supragingival tissue, a subgingival tissue, a tongue, a buccal mucosa, a soft palate, a hard palate, a floor of a mouth, or any other anatomical location of the oral cavity of the individual. In some embodiments, the oral biological sample is collected daily, weekly, monthly, or a combination thereof. In some embodiments, the machine learning algorithm is an unsupervised machine learning algorithm. In some embodiments, the machine learning algorithm is a supervised machine learning algorithm.

Disclosed herein, in certain embodiments, are methods of monitoring a progression of an oral and/or gum disease in an individual in need thereof, comprising: receiving a first plurality of sequence reads from a first oral biological sample of the individual and a second plurality of sequence read from a second oral biological sample of the individual, the first plurality of sequence reads and the second plurality of sequence reads corresponding to one or more nucleic acid molecules from at least one food source, wherein the first oral biological sample corresponds to a first collection time point and the second oral biological sample corresponds to a second collection time point; taxonomically classifying the first plurality of sequence reads and the second plurality of sequence reads to identify the at least one food source; quantifying the one or more nucleic acid molecules from the identified at least one food source, thereby generating a first calculated abundance and a second calculated abundance of the identified at least one food source; and applying a machine learning algorithm to: compare the first calculated abundance and the second calculated abundance to a cutoff abundance value associated with a healthy oral biological sample or with a diseased oral biological sample; calculate a first correlation value between the first calculated abundance and the cutoff abundance value; calculate a second correlation value between the second calculated abundance and the cutoff abundance value; compare the first correlation value to the second correlation value; and provide an indicator of the progression of the oral and/or gum disease in the individual based on the comparison between the first correlation value and the second correlation value, wherein the first calculated abundance and the second calculated abundance are used to continually retrain the machine learning algorithm to adjust the cutoff abundance value.

In some embodiments, the food source is a plant source, an animal source, or an edible fungal source. In some embodiments, the plant source is a plant from the kingdom of Thallophyta, Pteridophyta, or Spermatophyta. In some embodiments, the animal source is an invertebrate animal source or a vertebrate animal source. In some embodiments, the edible fungal source is a fungus from the Ascomycota phylum, Basidiomycota phylum, Blastocladiomycota phylum, Chytridiomycota phylum, Glomeromycota phylum, Microsporidia phylum, or Neocallimastigomycota phylum. In some embodiments, the oral biological sample comprises one or more nucleic acid molecules from a microorganism. In some embodiments, the microorganism is a bacterium, a virus, or a fungus. In some embodiments, the method further comprises applying steps a), b), and c) to the one or more nucleic acid molecules from the microorganism. In some embodiments, the calculated abundance of the at least one food source and/or a calculated abundance of the microorganism are used to estimate a pH value of the oral cavity of the individual. In some embodiments, the machine learning algorithm further compares the calculated abundance of the at least one food source and/or the calculated abundance of the microorganism to the pH value.

In some embodiments, the method further comprises receiving a survey datum from the individual, the survey datum being coincident with the oral biological sample. In some embodiments, the machine learning algorithm further converts the survey datum into a quantitative score. In some embodiments, the survey datum is used to continually retrain the machine learning algorithm to adjust the cutoff abundance value. In some embodiments, the calculated abundance is associated with the survey datum. In some embodiments, the survey datum comprises dietary information, medical history, medical records, lifestyle information, or any combinations thereof, of the individual. In some embodiments, the lifestyle information comprises information regarding alcohol use, tobacco use, dental hygiene habits, or any combination thereof, of the individual.

In some embodiments, the machine learning algorithm compares and correlates the calculated abundance to a gene expression profile of at least one gene in the oral biological sample. In some embodiments, the machine learning algorithm further compares the gene expression profile of the at least one gene in the oral biological sample to a reference gene expression profile of the at least one gene. In some embodiments, the at least one gene is differentially expressed in individuals who have an oral and/or gum disease as compared to individuals who do not have an oral and/or gum disease. In some embodiments, the reference gene expression profile is an average expression profile of the at least one gene in an oral biological sample of individuals who do not have an oral and/or gum disease. In some embodiments, the at least one gene is selected from MMP 10, MMP14, MMP16, MPPED2, ACTN2, VDR, FccRIIA, IL1-alpha, and IL1-beta.

In some embodiments, the machine learning algorithm is a random forest model, a t-distributed tSNE model, an artificial neural network model, a decision tree model, a kNN model, a PCA model, a TCA classifier, a deep neural network model, a support vector machine model, or a linear classification model. In some embodiments, the machine learning algorithm recognizes an abundance pattern in the calculated abundance. In some embodiments, the machine learning algorithm recognizes an abundance pattern in a calculated abundance of the microorganism. In some embodiments, the first oral biological sample and the second oral biological sample are a saliva sample, a biofilm sample, a dental tissue sample, a dental plaque sample, a tartar sample, a dental calculus sample, or any combination thereof.

In some embodiments, the first oral biological sample and the second oral biological sample are collected from: a supragingival tissue, a subgingival tissue, a tongue, a buccal mucosa, a soft palate, a hard palate, a floor of a mouth, or any other anatomical location of the oral cavity of the individual. In some embodiments, the first collection time point occurs one hour, six hours, twelve hours, twenty four hours, forty eight hours, seventy two hours, one week, four weeks, three months, six months, or twelve months prior to the second collection time point. In some embodiments, the individual is administered an oral and/or gum disease treatment after the first oral biological sample is collected. In some embodiments, the oral and/or gum disease treatment is a dental cleaning, a periodontal surgery, a gingival surgery, an antibiotic, a mouth wash, a fluoride treatment, a preventive restoration, an endodontic treatment, radiotherapy, chemotherapy, a biopsy, or any combination thereof.

In some embodiments, a response to the oral and/or gum disease treatment is measured based on the indicator of the progression of the oral and/or gum disease. In some embodiments, the machine learning algorithm is an unsupervised machine learning algorithm. In some embodiments, the machine learning algorithm is a supervised machine learning algorithm.

Disclosed herein, in certain embodiments, are computer-implemented systems for analyzing an oral biological sample from an individual, the oral biological sample comprising one or more nucleic acid molecules from at least one food source, the system comprising: a digital processing device, comprising: at least one processor, an operating system configured to perform executable instructions, a memory, and a computer program including the instructions executable by the digital processing device, the instructions comprising: receiving a plurality of sequence reads corresponding to the one or more nucleic acid molecules from the at least one food source; taxonomically classifying the plurality of sequence reads to identify the at least one food source; quantifying the one or more nucleic acid molecules from the identified at least one food source, thereby generating a calculated abundance of the identified at least one food source; applying a machine learning algorithm to: compare the calculated abundance to a cutoff abundance value associated with a healthy oral biological sample or with a diseased oral biological sample; calculate a correlation value between the calculated abundance and the cutoff abundance value; provide an indicator of an oral and/or gum disease in the individual based on the correlation value, and wherein the calculated abundance is used to continually retrain the machine learning algorithm to adjust the cutoff abundance value.

In some embodiments, the at least one food source is a plant source, an animal source, or an edible fungal source. In some embodiments, the plant source is a plant from the kingdom of Thallophyta, Pteridophyta, or Spermatophyta. In some embodiments, the animal source is an invertebrate animal source or a vertebrate animal source. In some embodiments, the edible fungal source is a fungus from the Ascomycota phylum, Basidiomycota phylum, Blastocladiomycota phylum, Chytridiomycota phylum, Glomeromycota phylum, Microsporidia phylum, or Neocallimastigomycota phylum. In some embodiments, the oral biological sample comprises one or more nucleic acid molecules from a microorganism. In some embodiments, the microorganism is a bacterium, a virus, or a fungus. In some embodiments, the instructions further comprise applying steps a), b), and c) to the one or more nucleic acid molecules from the microorganism.

In some embodiments, the calculated abundance of the at least one food source and/or a calculated abundance of the microorganism are used to estimate a pH value of the oral cavity of the individual. In some embodiments, the machine learning algorithm further compares the calculated abundance of the at least one food source and/or the calculated abundance of the microorganism to the pH value. In some embodiments, the instructions further comprise receiving a survey datum from the individual, the survey datum being coincident with the oral biological sample. In some embodiments, the machine learning algorithm further converts the survey datum into a quantitative score. In some embodiments, the survey datum is used to continually retrain the machine learning algorithm to adjust the cutoff abundance value. In some embodiments, the calculated abundance is associated with the survey datum. In some embodiments, the survey datum comprises dietary information, medical history, medical records, lifestyle information, or any combinations thereof, of the individual.

In some embodiments, the lifestyle information comprises information regarding alcohol use, tobacco use, dental hygiene habits, or any combination thereof, of the individual. In some embodiments, the machine learning algorithm compares and correlates the calculated abundance to a gene expression profile of at least one gene in the oral biological sample. In some embodiments, the machine learning algorithm further compares the gene expression profile of the at least one gene in the oral biological sample to a reference gene expression profile of the at least one gene. In some embodiments, the at least one gene is differentially expressed in individuals who have an oral and/or gum disease as compared to individuals who do not have an oral and/or gum disease. In some embodiments, the reference gene expression profile is an average expression profile of the at least one gene in an oral biological sample of individuals who do not have an oral and/or gum disease. In some embodiments, the at least one gene is selected from MMP 10, MMP14, MMP16, MPPED2, ACTN2, VDR, FccRIIA, IL1-alpha, and IL1-beta.

In some embodiments, the machine learning algorithm is a random forest model, a tSNE model, an artificial neural network model, a decision tree model, a kNN model, a PCA model, a TCA classifier, a deep neural network model, a support vector machine model, or a linear classification model. In some embodiments, the machine learning algorithm recognizes an abundance pattern in the calculated abundance. In some embodiments, the machine learning algorithm recognizes an abundance pattern in a calculated abundance of the microorganism. In some embodiments, the oral biological sample is a saliva sample, a biofilm sample, a dental tissue sample, a dental plaque sample, a tartar sample, a dental calculus sample, or any combination thereof. In some embodiments, the oral biological sample is collected from: a supragingival tissue, a subgingival tissue, a tongue, a buccal mucosa, a soft palate, a hard palate, a floor of a mouth, or any other anatomical location of the oral cavity of the individual. In some embodiments, the oral biological sample is collected daily, weekly, monthly, or a combination thereof.

In one aspect, disclosed herein are systems for providing a recommendation for an individual regarding oral health based on an analysis of an oral biological sample from the individual comprising a computing device comprising a user interface; at least one processor, and a computer-readable storage device coupled to the at least one processor and having instructions stored thereon which, when executed by the at least one processor, cause the at least one processor to perform operations comprising: receiving a plurality of sequence reads of the oral biological sample collected from the individual, wherein each of the sequence reads corresponding to one or more nucleic acid molecules from at least one food source or at least one microorganism; determining an abundance of the at least one food source or the at least one microorganism by: taxonomically classifying the sequence reads to identify the at least one food source or the at least one microorganism; and quantifying the one or more nucleic acid molecules from the identified at least one food source or the at least one microorganism; processing the abundance of the at least one food source or the at least one microorganism through a machine learning algorithm to determine an indicator of an oral or gum disease in the individual, the machine learning algorithm having been trained using a plurality previously received sequence reads of oral biological samples collected from other individuals; and providing, to the user interface, the recommendation for the individual regarding oral health based on the indicator of the oral or gum disease in the individual. In some embodiments, the indicator of an oral or gum disease in the individual is determined based on a correlation value between the abundance of the at least one food source or the at least one microorganism and a cutoff abundance value. In some embodiments, the operations comprise: retraining the machine learning algorithm with the abundance of the at least one food source or the at least one microorganism to adjust the cutoff abundance value. In some embodiments, the cutoff abundance value is associated with a healthy oral biological sample, a diseased oral biological sample, or a risk or probability of disease. In some embodiments, the oral biological samples collected from the other individuals comprise the healthy oral biological sample and the diseased oral biological sample. In some embodiments, the operations comprise: receiving a survey datum from the individual, the survey datum being coincident with the oral biological sample processing the survey datum through the machine learning algorithm to determine a quantitative score. In some embodiments, the operations comprise: retraining the machine learning algorithm with survey datum to adjust the cutoff abundance value, wherein the abundance is associated with the survey datum. In some embodiments, the survey datum comprises dietary information, medical history, medical records, lifestyle information, or any combinations thereof, of the individual. In some embodiments, the operations comprise: receiving a gene expression profile of at least one gene in the oral biological sample collected from the individual; and processing the gene expression profile through the machine learning algorithm to determine the indicator of an oral or gum disease in the individual. In some embodiments, the indicator of an oral or gum disease in the individual is determined based on a correlation value between the gene expression profile and a reference gene expression profile of the at least one gene, wherein the reference gene expression profile is determined based on the expression profiles of the at least one gene in oral biological samples of other individuals who do not have an oral and/or gum disease. In some embodiments, the at least one gene is differentially expressed in individuals who have an oral and/or gum disease as compared to individuals who do not have an oral and/or gum disease. In some embodiments, the at least one gene is selected from MMP 10, MMP14, MMP16, MPPED2, ACTN2, VDR, FccRIIA, IL1-alpha, and IL1-beta. In some embodiments, the operations comprise: processing the abundance of the at least one food source or the at least one microorganism through a machine learning algorithm to determine a pH value of an oral cavity of the individual, wherein the indicator of an oral or gum disease in the individual is determined based on the pH value of the oral cavity of the individual, and wherein the recommendation for the individual regarding oral health is determined based on the pH value of the oral cavity of the individual. In some embodiments, the oral biological sample comprises a saliva sample, a biofilm sample, a dental tissue sample, a dental plaque sample, a tartar sample, a dental calculus sample, or any combination thereof. In some embodiments, the oral biological sample is collected from: a supragingival tissue, a subgingival tissue, a tongue, a buccal mucosa, a soft palate, a hard palate, a floor of a mouth, or any other anatomical location of the oral cavity of the individual. In some embodiments, the machine learning algorithm comprises a random forest model, a tSNE model, an artificial neural network model, a decision tree model, a kNN model, a PCA model, a TCA classifier, a deep neural network model, a support vector machine model, or a linear classification model. In some embodiments, the at least one food source comprises a plant source, an animal source, or an edible fungal source, and wherein the at least one microorganism comprises a bacterium, a virus, or a fungus. In some embodiments, the recommendation for the individual regarding oral health comprises prebiotics, probiotics, or other diet components to shift a pH of an oral cavity of the individual.

In another aspect, disclosed herein are computer-implemented methods for providing a recommendation for an individual regarding oral health based on an analysis of an oral biological sample from the individual comprising: receiving a plurality of sequence reads of the oral biological sample collected from the individual, wherein each of the sequence reads corresponding to one or more nucleic acid molecules from at least one food source or at least one microorganism; determining an abundance of the at least one food source or the at least one microorganism by: taxonomically classifying the sequence reads to identify the at least one food source or the at least one microorganism; and quantifying the one or more nucleic acid molecules from the identified at least one food source or the at least one microorganism; processing the abundance of the at least one food source or the at least one microorganism through a machine learning algorithm to determine an indicator of an oral or gum disease in the individual, the machine learning algorithm having been trained using a plurality previously received sequence reads of oral biological samples collected from other individuals; and providing, to a user interface, the recommendation for the individual regarding oral health based on the indicator of the oral or gum disease in the individual. In some embodiments, the indicator of an oral or gum disease in the individual is determined based on a correlation value between the abundance of the at least one food source or the at least one microorganism and a cutoff abundance value. In some embodiments, the methods comprise: retraining the machine learning algorithm with the abundance of the at least one food source or the at least one microorganism to adjust the cutoff abundance value. In some embodiments, the cutoff abundance value is associated with a healthy oral biological sample, a diseased oral biological sample, or a risk or probability of disease. In some embodiments, the oral biological samples collected from the other individuals comprise the healthy oral biological sample and the diseased oral biological sample. In some embodiments, the methods comprise: receiving a survey datum from the individual, the survey datum being coincident with the oral biological sample processing the survey datum through the machine learning algorithm to determine a quantitative score. In some embodiments, the methods comprise: retraining the machine learning algorithm with survey datum to adjust the cutoff abundance value, wherein the abundance is associated with the survey datum. In some embodiments, the survey datum comprises dietary information, medical history, medical records, lifestyle information, or any combinations thereof, of the individual. In some embodiments, the methods comprise: receiving a gene expression profile of at least one gene in the oral biological sample collected from the individual; and processing the gene expression profile through the machine learning algorithm to determine the indicator of an oral or gum disease in individual. In some embodiments, the indicator of an oral or gum disease in the individual is determined based on a correlation value between the gene expression profile and a reference gene expression profile of the at least one gene, wherein the reference gene expression profile is determined based on the expression profiles of the at least one gene in oral biological samples of other individuals who do not have an oral and/or gum disease. In some embodiments, the at least one gene is differentially expressed in individuals who have an oral and/or gum disease as compared to individuals who do not have an oral and/or gum disease. In some embodiments, the at least one gene is selected from MMP 10, MMP14, MMP16, MPPED2, ACTN2, VDR, FccRIIA, IL1-alpha, and IL1-beta. In some embodiments, the methods comprise: processing the abundance of the at least one food source or the at least one microorganism through a machine learning algorithm to determine a pH value of an oral cavity of the individual, wherein the indicator of an oral or gum disease in the individual is determined based on the pH value of the oral cavity of the individual, and wherein the recommendation for the individual regarding oral health is determined based on the pH value of the oral cavity of the individual. In some embodiments, the oral biological sample comprises a saliva sample, a biofilm sample, a dental tissue sample, a dental plaque sample, a tartar sample, a dental calculus sample, or any combination thereof. In some embodiments, the oral biological sample is collected from: a supragingival tissue, a subgingival tissue, a tongue, a buccal mucosa, a soft palate, a hard palate, a floor of a mouth, or any other anatomical location of the oral cavity of the individual. In some embodiments, the machine learning algorithm comprises a random forest model, a tSNE model, an artificial neural network model, a decision tree model, a kNN model, a PCA model, a TCA classifier, a deep neural network model, a support vector machine model, or a linear classification model. In some embodiments, the at least one food source comprises a plant source, an animal source, or an edible fungal source, and wherein the at least one microorganism comprises a bacterium, a virus, or a fungus. In some embodiments, the recommendation for the individual regarding oral health comprises prebiotics, probiotics, or other diet components to shift a pH of an oral cavity of the individual.

In another aspect, disclosed herein are computer-implemented methods for monitoring a progression of an oral and/or gum disease in an individual in need thereof comprising: receiving a first plurality of sequence reads from a first oral biological sample of the individual and a second plurality of sequence reads from a second oral biological sample of the individual, the first plurality of sequence reads and the second plurality of sequence reads corresponding to one or more nucleic acid molecules from at least one food source, wherein the first oral biological sample corresponds to a first collection time point and the second oral biological sample corresponds to a second collection time point; determining a first calculated abundance and a second calculated abundance of the at least one food source by: taxonomically classifying the first plurality of sequence reads and the second plurality of sequence reads to identify the at least one food source; and quantifying the one or more nucleic acid molecules from the identified at least one food source; processing the first calculated abundance and the second calculated abundance of the at least one food source through a machine learning algorithm to determine the progression of the oral or gum disease in the individual, the machine learning algorithm having been trained using a plurality previously received sequence reads of oral biological samples collected from other individuals; and providing, to a user interface, an indication regarding the progression of the oral or gum disease in the individual. In some embodiments, the progression of an oral or gum disease in the individual is determined based on a correlation value between the first calculated abundance, the second calculated abundance of the at least one food source, a cutoff abundance value. In some embodiments, the methods comprise: retraining the machine learning algorithm with the first calculated abundance, the second calculated abundance of the at least one food source to adjust the cutoff abundance value. In some embodiments, the cutoff abundance value is associated with a healthy oral biological sample, a diseased oral biological sample, or a risk or probability of disease. In some embodiments, the oral biological samples collected from the other individuals comprise the healthy oral biological sample and the diseased oral biological sample. In some embodiments, the methods comprise: receiving a survey datum from the individual, the survey datum being coincident with the oral biological sample processing the survey datum through the machine learning algorithm to determine a quantitative score. In some embodiments, the methods comprise: retraining the machine learning algorithm with survey datum to adjust the cutoff abundance value, wherein the abundance is associated with the survey datum. In some embodiments, the survey datum comprises dietary information, medical history, medical records, lifestyle information, or any combinations thereof, of the individual. In some embodiments, the methods comprise: receiving a gene expression profile of at least one gene in the first or second oral biological samples collected from the individual; and processing the gene expression profile through the machine learning algorithm to determine the progression of an oral or gum disease in the first or second oral biological sample. In some embodiments, the progression of an oral or gum disease in the individual is determined based on a correlation value between the gene expression profile and a reference gene expression profile of the at least one gene, wherein the reference gene expression profile is determined based on the expression profiles of the at least one gene in first or second oral biological samples of other individuals who do not have an oral and/or gum disease. In some embodiments, the at least one gene is differentially expressed in individuals who have an oral and/or gum disease as compared to individuals who do not have an oral and/or gum disease. In some embodiments, the at least one gene is selected from MMP 10, MMP14, MMP16, MPPED2, ACTN2, VDR, FccRIIA, IL1-alpha, and IL1-beta. In some embodiments, the methods comprise: processing the abundance of the at least one food source through a machine learning algorithm to determine a pH value of an oral cavity of the individual, wherein the progression of an oral or gum disease in the individual is determined based on the pH value of the oral cavity of the individual, and wherein the recommendation for the individual regarding oral health is determined based on the pH value of the oral cavity of the individual. In some embodiments, the first or second oral biological samples comprises a saliva sample, a biofilm sample, a dental tissue sample, a dental plaque sample, a tartar sample, a dental calculus sample, or any combination thereof. In some embodiments, the first or second oral biological samples is collected from: a supragingival tissue, a subgingival tissue, a tongue, a buccal mucosa, a soft palate, a hard palate, a floor of a mouth, or any other anatomical location of the oral cavity of the individual. In some embodiments, the machine learning algorithm comprises a random forest model, a tSNE model, an artificial neural network model, a decision tree model, a kNN model, a PCA model, a TCA classifier, a deep neural network model, a support vector machine model, or a linear classification model. In some embodiments, the at least one food source comprises a plant source, an animal source, or an edible fungal source.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer control system that is programmed or otherwise configured to implement methods provided herein.

FIG. 2 shows an exemplary flow chart illustrating a method provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” refers to an amount that is near the stated amount by about 10%, 5%, or 1%, including increments therein. For example, “about” or “approximately” can mean a range including the particular value and ranging from 10% below that particular value and spanning to 10% above that particular value.

As used herein, the term “biological sample,” refers to any suitable biological or contrived sample (for example, cells in centrifugation media) that comprises a nucleic acid, a protein, or any other biological analyte. The biological sample may be obtained from a subject. A biological sample may be solid matter (e.g., biological tissue) or may be a fluid (e.g., a biological fluid). In general, a biological fluid can include any fluid associated with living organisms. Non-limiting examples of a biological sample include saliva, a biofilm, dental tissue, dental plaque, tartar, a dental calculus, or a dental sample obtained from any anatomical location of the oral cavity of a subject; cells obtained from any anatomical location of the oral cavity of the subject; throat swab; oral biopsy; an oral cavity fluid; sputum; pus; microbiota; and/or other oral tissues.

A biological sample may be obtained from a subject by any means known in the art. Non-limiting examples of means to obtain a biological sample directly from a subject include accessing the oral cavity (e.g., via dental prophylaxis, manipulation of supragingival tissue, manipulation of subgingival tissue, curette scraping, endodontic paper point, root planing, and/or scaling), collecting a secreted biological sample (e.g., sputum, saliva, etc.), surgically (e.g., biopsy), swabbing (e.g., buccal swab, oropharyngeal swab), and pipetting. Moreover, a biological sample may be obtained from any anatomical part of a subject where a desired biological sample is located. Alternatively, a sample can be constructed by mixing biological and non-biological substances.

As used herein, the term “subject,” generally refers to an entity or a medium that has testable or detectable biological information. A biological sample can be obtained from a subject. A subject can be a person, an individual, or a patient. A subject can be an invertebrate or a vertebrate, such as, for example, a mammal. Non-limiting examples of mammals include murine mammals, simians, humans, farm animals, sport animals, and pets.

As used herein, the term a “nucleic acid sample” refers to a collection of nucleic acid molecules. In some instances, the nucleic acid sample may be from a single biological source, e.g. one individual or one tissue sample, and in other instances, the nucleic acid sample may be a pooled sample, e.g., containing nucleic acids from more than one organism, individual or tissue. In some instances, the nucleic acid sample may be a recombinant nucleic acid. Non-limiting examples of synthetic nucleic acids include plasmids, viral vectors, and short hairpin ribonucleic acid (shRNAs). In some instances, the nucleic acid sample may be a synthetic nucleic acid. Non-limiting examples of synthetic nucleic acids include synthetic ribonucleic acid (RNA) such as RNA spike-ins, synthetic DNA such as sequins, primers, modified analogs of nucleotides such as morpholinos, and small interfering ribonucleic acid (siRNA).

As used herein, the term “collected data” refers to the oral biological sample collected, the plurality of sequence reads of the oral biological sample, and/or the survey datum collected from the individual.

Human Microbiomes

The human body contains a personalized microbiome that plays an important role in health and disease. In particular, the oral microbiome is of great significance because it has the capability of triggering both oral and systemic diseases. The oral microbiome is defined as the collective genomes of the microorganisms (i.e., the microbiota) residing in biofilms or in planktonic state throughout the oral cavity. The oral microbiota comprises a number of different types of ecological niches within the oral cavity that change throughout the life of an individual. Non-limiting examples of such ecological niches include supragingival plaque, subgingival plaque, saliva, and buccal mucosa. These oral microbiota form an ecosystem that when disrupted, leads to a disease state. Furthermore, the collective genomes of food source remnants (e.g., plant, fungus, and animal sources) present in the oral cavity provide valuable information regarding the diet of an individual. The systems and methods provided herein analyze oral biological samples comprising one or more nucleic acid molecules from at least one food source. The systems and methods provided herein describe different methodologies to identify life style practices, supplements, diet elements, or other intervention strategies that improve oral health. Also described herein are methods and systems for oral health monitoring, oral health treatment suggestions, and measuring oral disease progression.

Methods of Analyzing Oral Biological Samples

Disclosed herein, in certain embodiments, are methods of predicting an oral and/or gum disease in an individual in need thereof. Additionally disclosed herein, in certain embodiments, are methods of monitoring a progression of an oral and/or gum disease in an individual in need thereof.

In some embodiments, the oral and/or gum disease is a dental cavity or a dental caries. In some embodiments, the oral and/or gum disease is an infectious disease such as oral herpes. In some embodiment the collected data can be used to identify following viruses that may affect oral tissues: Picornaviridae, Varicella-Zoster, Cytomegalovirus an Epstein-Barr virus, Human Papillomaviruses. In some embodiment the collected data can be used to identify viruses present in oral cavity: Hepatitis B virus, Hepatitis D virus, Hepatitis C virus, Human Immunodeficiency virus (HIV). In some embodiments, the oral and/or gum disease is a periodontal disease. In some embodiments, the periodontal disease is gingivitis, periodontitis, a periodontal abscess, a perio-endo lesion, a gingival recession. In some embodiments, the periodontitis is chronic periodontitis or aggressive periodontitis. In some embodiments, the oral and/or gum disease is necrotizing, ulcerative gingivitis. In some embodiments, the oral or gum disease is tooth decay. In some embodiments, the oral or gum disease is oral cancer. In some embodiments, the oral and/or gum disease is leukoplakia. In some embodiments, the oral and/or gum disease is plaque. In some embodiments, the oral and/or gum disease is calculus. In some embodiments, the oral and/or gum disease is a tooth infection. In some embodiments, the oral and/or gum disease is tooth erosion. In some embodiments, the oral and/or gum disease is tooth sensitivity. In some embodiments, the oral and/or gum disease is noma.

In some embodiments, the methods comprise a) receiving a plurality of sequence reads from an oral biological sample of the individual (see step 202 in FIG. 2). In some embodiments, the oral biological sample is a saliva sample, a biofilm sample, a dental tissue sample, a dental plaque sample, a tartar sample, a dental calculus sample, or any combination thereof. In some embodiments, the oral biological sample is collected from: a supragingival tissue, a subgingival tissue, a tongue, a buccal mucosa, a soft palate, a hard palate, a floor of a mouth, or any other anatomical location of the oral cavity of the individual. In some embodiments, the oral biological sample is collected through sampling of different locations of an individual's oral cavity, either a healthy spot or any region impacted by an oral or gum disease. In some embodiments, the sampled spot is exposed. In some embodiments, the sampled spot is buried under the patient's gums, hence less accessible.

In some embodiments, the oral biological sample is collected daily, weekly, monthly, or a combination thereof. In some embodiments, the oral biological sample is collected at least about two times in 1 day. In some embodiments, the oral biological sample is collected at least about three times in 1 day. In some embodiments, the oral biological sample is collected daily for a period of about at least 1 day to about 28 days or more. In some embodiments, the oral biological sample is collected daily for a period of at least about 1 day. In some embodiments, the oral biological sample is collected daily for a period of at most about 28 days. In some embodiments, the oral biological sample is collected daily for a period of about 1 day to about 2 days, about 1 day to about 3 days, about 1 day to about 4 days, about 1 day to about 5 days, about 1 day to about 6 days, about 1 day to about 7 days, about 1 day to about 14 days, about 1 day to about 21 days, about 1 day to about 28 days, about 2 days to about 3 days, about 2 days to about 4 days, about 2 days to about 5 days, about 2 days to about 6 days, about 2 days to about 7 days, about 2 days to about 14 days, about 2 days to about 21 days, about 2 days to about 28 days, about 3 days to about 4 days, about 3 days to about 5 days, about 3 days to about 6 days, about 3 days to about 7 days, about 3 days to about 14 days, about 3 days to about 21 days, about 3 days to about 28 days, about 4 days to about 5 days, about 4 days to about 6 days, about 4 days to about 7 days, about 4 days to about 14 days, about 4 days to about 21 days, about 4 days to about 28 days, about 5 days to about 6 days, about 5 days to about 7 days, about 5 days to about 14 days, about 5 days to about 21 days, about 5 days to about 28 days, about 6 days to about 7 days, about 6 days to about 14 days, about 6 days to about 21 days, about 6 days to about 28 days, about 7 days to about 14 days, about 7 days to about 21 days, about 7 days to about 28 days, about 14 days to about 21 days, about 14 days to about 28 days, or about 21 days to about 28 days. In some embodiments, the oral biological sample is collected daily for a period of about 1 day, about 2 days, about 3 days, about 4 days, about 5 days, about 6 days, about 7 days, about 14 days, about 21 days, or about 28 days.

In some embodiments, the oral biological sample is collected weekly for a period of at least about 1 week to about 520 weeks or more. In some embodiments, the oral biological sample is collected weekly for a period of at least about 1 week. In some embodiments, the oral biological sample is collected weekly for a period of at most about 520 weeks. In some embodiments, the oral biological sample is collected weekly for a period of about 1 week to about 2 weeks, about 1 week to about 3 weeks, about 1 week to about 4 weeks, about 1 week to about 6 weeks, about 1 week to about 12 weeks, about 1 week to about 24 weeks, about 1 week to about 52 weeks, about 1 week to about 104 weeks, about 1 week to about 260 weeks, about 1 week to about 520 weeks, about 2 weeks to about 3 weeks, about 2 weeks to about 4 weeks, about 2 weeks to about 6 weeks, about 2 weeks to about 12 weeks, about 2 weeks to about 24 weeks, about 2 weeks to about 52 weeks, about 2 weeks to about 104 weeks, about 2 weeks to about 260 weeks, about 2 weeks to about 520 weeks, about 3 weeks to about 4 weeks, about 3 weeks to about 6 weeks, about 3 weeks to about 12 weeks, about 3 weeks to about 24 weeks, about 3 weeks to about 52 weeks, about 3 weeks to about 104 weeks, about 3 weeks to about 260 weeks, about 3 weeks to about 520 weeks, about 4 weeks to about 6 weeks, about 4 weeks to about 12 weeks, about 4 weeks to about 24 weeks, about 4 weeks to about 52 weeks, about 4 weeks to about 104 weeks, about 4 weeks to about 260 weeks, about 4 weeks to about 520 weeks, about 6 weeks to about 12 weeks, about 6 weeks to about 24 weeks, about 6 weeks to about 52 weeks, about 6 weeks to about 104 weeks, about 6 weeks to about 260 weeks, about 6 weeks to about 520 weeks, about 12 weeks to about 24 weeks, about 12 weeks to about 52 weeks, about 12 weeks to about 104 weeks, about 12 weeks to about 260 weeks, about 12 weeks to about 520 weeks, about 24 weeks to about 52 weeks, about 24 weeks to about 104 weeks, about 24 weeks to about 260 weeks, about 24 weeks to about 520 weeks, about 52 weeks to about 104 weeks, about 52 weeks to about 260 weeks, about 52 weeks to about 520 weeks, about 104 weeks to about 260 weeks, about 104 weeks to about 520 weeks, or about 260 weeks to about 520 weeks. In some embodiments, the oral biological sample is collected weekly for a period of about 1 week, about 2 weeks, about 3 weeks, about 4 weeks, about 6 weeks, about 12 weeks, about 24 weeks, about 52 weeks, about 104 weeks, about 260 weeks, or about 520 weeks.

In some embodiments, the oral biological sample is collected monthly for a period of at least about 1 month to about 120 months or more. In some embodiments, the oral biological sample is collected monthly for a period of at least about 1 month. In some embodiments, the oral biological sample is collected monthly for a period of at most about 120 months. In some embodiments, the oral biological sample is collected monthly for a period of about 1 month to about 2 months, about 1 month to about 3 months, about 1 month to about 6 months, about 1 month to about 12 months, about 1 month to about 24 months, about 1 month to about 36 months, about 1 month to about 48 months, about 1 month to about 60 months, about 1 month to about 120 months, about 2 months to about 3 months, about 2 months to about 6 months, about 2 months to about 12 months, about 2 months to about 24 months, about 2 months to about 36 months, about 2 months to about 48 months, about 2 months to about 60 months, about 2 months to about 120 months, about 3 months to about 6 months, about 3 months to about 12 months, about 3 months to about 24 months, about 3 months to about 36 months, about 3 months to about 48 months, about 3 months to about 60 months, about 3 months to about 120 months, about 6 months to about 12 months, about 6 months to about 24 months, about 6 months to about 36 months, about 6 months to about 48 months, about 6 months to about 60 months, about 6 months to about 120 months, about 12 months to about 24 months, about 12 months to about 36 months, about 12 months to about 48 months, about 12 months to about 60 months, about 12 months to about 120 months, about 24 months to about 36 months, about 24 months to about 48 months, about 24 months to about 60 months, about 24 months to about 120 months, about 36 months to about 48 months, about 36 months to about 60 months, about 36 months to about 120 months, about 48 months to about 60 months, about 48 months to about 120 months, or about 60 months to about 120 months. In some embodiments, the oral biological sample is collected monthly for a period of about 1 month, about 2 months, about 3 months, about 6 months, about 12 months, about 24 months, about 36 months, about 48 months, about 60 months, or about 120 months.

In some embodiments, the nucleic acid molecules of the oral biological sample are sequenced. In some embodiments, next generation sequencing (NGS) or any other massively parallelized nucleic acid characterization approach is used to identify the composition of the oral biological sample (i.e., the biomass). In some embodiments, the nucleic acid molecules of the oral biological sample are sequenced by any suitable sequencing methods known in the art. For example, in some embodiments, the nucleic acid molecules are sequenced using sequencing methods comprising shotgun sequencing and/or bridge polymerase chain reaction (PCR). In some embodiments, the nucleic acid molecules are sequenced using high throughput sequencing methods. In some embodiments, the high throughput sequencing methods comprise massively parallel signature sequencing (MPSS), polony sequencing, 454 pyrosequencing, Illumina (Solexa) sequencing, SOLiD sequencing, ion torrent sequencing, DNA nanoball sequencing, heliscope single molecule sequencing, single molecule real time (SMT) sequencing, nanopore DNA sequencing, or any combination thereof. In some embodiments, the nucleic acid molecules are sequenced using PCR-free sequencing methods. In some embodiments, the nucleic acid molecules are sequenced using whole genome sequencing (WGS). In some embodiments, sequencing the nucleic acid molecules of the oral biological sample yields a plurality of sequence reads.

In some embodiments, the nucleic acid characterization comprises the use of shotgun sequencing or targeted sequencing. In some embodiments, the oral biological samples are characterized by alternative approaches (i.e., non-genomic sequencing methods) including, but not limited to array- or bead-based hybridization, mass spectrophotometry, or mass spectrometry. In some embodiments, the oral biological samples are processed in a manner that allows characterizing all the biological components present within the sample including but not limited to animal, plant, bacterial, and fungal components. In some embodiments, if the oral biological sample is characterized via nucleic acid sequencing, signature sequences from bacterial, viral, and fungal components are used to assemble the microbiome of the analyzed sample. In some embodiments, the oral biological samples are characterized by looking at methylation or other epigenetic patterns. In some embodiments, signature sequences from animal, plant, and edible fungal components are used to infer the individual's diet.

In some embodiments, the plurality of sequence reads corresponds to one or more nucleic acid molecules from at least one food source. In some embodiments, the at least one food source originates from the individual's dietary consumption. In some embodiments, the oral biological sample contains food remnants or traces of food that are later identified via sequencing. In some embodiments, the methods comprise b) taxonomically classifying the plurality of sequence reads to identify the at least one food source (see step 204 in FIG. 2). In some embodiments, the at least one food source is a plant source, an animal source, or an edible fungal source. In some embodiments, the plant source is a plant from the kingdom of Thallophyta, Pteridophyta, or Spermatophyta. In some embodiments, the plant source is a vegetable, a fruit, a legume, a grain, a seed, a nut, a spice, a condiment, a dietary supplement, algae, or any combination thereof. In some embodiments, the animal source is an invertebrate animal source or a vertebrate animal source. In some embodiments, the animal source is seafood, beef, poultry, eggs, pork, lamb, mutton, venison, a dietary supplement, or any combination thereof. In some embodiments, the animal source is a highly processed meat. In some embodiments, the edible fungal source is a fungus from the Ascomycota phylum, Basidiomycota phylum, Blastocladiomycota phylum, Chytridiomycota phylum, Glomeromycota phylum, Microsporidia phylum, or Neocallimastigomycota phylum. In some embodiments, the at least one food source is an artificial sweetener. Non-limiting examples of artificial sweeteners include acesulfame potassium, aspartame, aspartame-acesulfame salt, cyclamate, erythrol, glycerol, glycyrrhizin, hydrogenated starch hydrolysate (HSH), isomalt, lactitol, malitol, mannitol, neotame, polydextrose, saccharin, sorbitol, steviol glycoside, sucralose, tagatose, and xylitol. In some embodiments, the at least one food source is a synthetic food or an artificial food. In some embodiments, the synthetic food is a dietary supplement or a vitamin.

In some embodiments, the methods comprise c) quantifying the one or more nucleic acid molecules from the identified at least one food source, thereby generating a calculated abundance of the identified at least one food source (see step 206 in FIG. 2). In some embodiments, quantifying the one or more nucleic acid molecules comprises organizing the plurality of sequences into one or more operational taxonomic units. An operational taxonomical unit (OTU), as used herein, refers to clusters of organisms, grouped based on DNA sequence similarity of one or more taxonomic marker genes. In some embodiments, an OTU is a proxy for a “species.” In some embodiments, the taxonomic marker gene is a small subunit 16S rRNA marker gene or an 18S rRNA marker gene.

In some embodiments, the methods comprise d) applying a machine learning algorithm (see step 208 in FIG. 2). In some embodiments, the machine learning algorithm compares the calculated abundance to a cutoff abundance value associated with a healthy oral biological sample or with a diseased oral biological sample (see step 210 in FIG. 2). In some embodiments, the cutoff abundance value is associated with a risk or probability of disease. In some embodiments, the machine learning algorithm calculates a correlation value between the calculated abundance and the cutoff abundance value. In some embodiments, the machine learning algorithm provides an indicator of an oral and/or gum disease in the individual based on the correlation value (see step 210 in FIG. 2). In some embodiments, the calculated abundance is used to continually retrain the machine learning algorithm to adjust the cutoff abundance value. In some embodiments, a plurality of calculated abundances is used to continually retrain the machine learning algorithm. In some embodiments, metadata from a patient is used to continually retrain the machine learning algorithm. In some embodiments, a second oral biological sample is obtained from a patient at a time later than a first oral biological sample. In some embodiments, the first oral biological sample and the second biological sample are used to continually retrain the machine learning algorithm. In some embodiments, in this case, the second biological sample is reflective of a future evolution of the condition of the patient. For example, in some embodiments, the patient has a disease and the second oral biological sample is reflective of the progression of the disease. In some embodiments, the machine learning algorithm is retrained using this new data. In some embodiments, the disease has progressed further at the time the second oral biological sample is collected. In some embodiments, the disease has regressed at the time the second oral biological sample is collected. In some embodiments, the accuracy of the machine learning model is measured by applying the model to known patients being observed. In some embodiments, any deviation of the applied model from desired thresholds triggers a retraining of the model with more accumulated data (e.g., additional oral biological samples, additional calculated abundances, and/or additional metadata).

In some embodiments, the oral biological sample comprises one or more nucleic acid molecules from a microorganism. In some embodiments, the microorganism is a bacterium, a virus, or a fungus. In some embodiments, the oral biological sample comprises a bacteriophage. In some embodiments, the bacterial or fungal species is Streptococcus mutans, Streptococcus sobrinus, Porphyromonas gingivalis, Treponema denticola, Tannerella forsythia, Aggregatibacter actinomycetemcomitans, Fusobacterium nucleatum, Bacteroides species, Actinomyces species, Streptococcus intermedius, Candida albicans, Streptococcus sanguinis, Streptococcus oxalis, Streptococcus mitis bv.1, Streptococcus gordoni, Lactobacilli, Actinomyces naeslundii, Actinomyces viscosus, Selenomonas sputigena, Haemophilus parainfluenzae, Actinomyces israelii, Streptococcus mitis, Peptostreptococcus, Prevotella intermedia, Campylobacter sputorum, Veillonella species, TM4, Atopobium parvulum, Eubacterium species, Abiotrophia adiacnes, Dialister pneumosintes, Filifactor alocis, Selenomonas sp GAA14, Streptococcus constellatus, Bacteroides forsythus, Eubacterium nodatum, and/or Campylobacter rectus. In some embodiments, the bacterial or fungal species is a novel species or strain that is not disclosed supra. In some embodiments, the bacterial, fungal, or viral signature of the oral or gum disease is assessed based on depletion of the bacteria that are known to promote good oral health. In some embodiments, bacteria and/or fungi that are known to promote good oral health include but are not limited to Streptococcus dentistani, Streptococcus salivarius strain M18, Lactobacillus paracasei ssp. Paracasei, Lactobacillus rhamnosus, Weissella cibaria, Streptococcus thermophilus, Lactobacillus lactis spp. Lactis, Lactobacillus gasseri, Lactobacillus fermentum, Lactobacillus brevis, Lactobacillus reuteri, Streptococcus salivarius, Streptococcus salivarius strain K12, Lactobacillus salivarius T12711, Lactobacillu acidophilus, Lactobacillu casei, Lactobacillu crispatus, Lactobacillu delbrueckii subsp. bulgaricus, Lactobacillu fermentum, Lactobacillu gasseri, Lactobacillu johnsonii, Lactobacillu paracasei, Lactobacillu plantarum, Lactobacillu rhamnosus, Bifidobacterium bifidum, Bifidobacterium breve, Bifidobacterium infantis, Bifidobacterium longum, Bifidobacterium lactis, Bifidobacterium adolescentis, Bifidobacterium DN-173 010, Bifidobacterium animalis, Bifidobacterium thermophilum, Streptococcus faecium, Candida albicans, Saccharomyces boulardii, Aspergillus oryzae, Candida pintolopesii, Saccharomyces boulardii, Lactococcus lactis subsp. cremoris, Enterococcus faecium, Streptococcus diaacetylactis, Streptococcus intermediu, Streptococcus salivarius, Leuconostoc species, Pediococcus species, Propionibacterium species, Streptococci, Streptococcus mitis bv. 1, Streptococcus gordoni, Veillonellae, Streptococcus sanguinis, Streptococcus oralis, Actinomyces, Streptococcus mitis bv.2, and Streptococcus salivariu.

In some embodiments, the oral or gum disease is characterized by absence or shortage of health-associated bacteria including, but not limited to Streptococci, Streptococcus mitis bv. 1, Streptococcus gordoni, Veillonellae, Streptococcus sanguinis, Streptococcus oralis, Actinomyces, Streptococcus mitis bv.2, & Streptococcus salivarius.

In some embodiments, the methods further comprise applying steps a), b), and c) to the one or more nucleic acid molecules from the microorganism. In some embodiments, the calculated abundance of the at least one food source and/or a calculated abundance of the microorganism are used to estimate a pH value of the oral cavity of the individual. In some embodiments, the machine learning algorithm further compares the calculated abundance of the at least one food source and/or the calculated abundance of the microorganism to the pH value.

In some embodiments, the calculated abundance is used to identify a type of bacterium that contributes to oral pH. In some embodiments, the calculated abundance is used to identify a type of fungus that contributes to oral pH. In some embodiments, the calculated abundance is used to identify a type of virus that contributes to oral pH. In some embodiments, the calculated abundance is used to identify a type of bacteriophage that contributes to oral pH. In some embodiments, the calculated abundance is used to identify a type of food that contributes to oral pH. In some embodiments, the calculated abundance is used to identify a diet element that contributes to oral pH.

In some embodiments, the calculated abundance is used to identify a type of bacterium in combination with a type of food that contributes to oral pH. In some embodiments, the calculated abundance is used to identify a type of virus in combination with a type of food that contributes to oral pH. In some embodiments, the calculated abundance is used to identify a type of fungus in combination with a type of food that contributes to oral pH. In some embodiments, the calculated abundance is used to identify a type of bacteriophage in combination with a type of food that contributes to oral pH. In some embodiments, the calculated abundance is used to identify a type of bacterium in combination with a diet element that contributes to oral pH. In some embodiments, the calculated abundance is used to identify a type of virus in combination with a diet element that contributes to oral pH. In some embodiments, the calculated abundance is used to identify a type of fungus in combination with a diet element that contributes to oral pH. In some embodiments, the calculated abundance is used to identify a type of bacteriophage in combination with a diet element that contributes to oral pH.

In some embodiments, the methods further comprise metabolic modeling of the oral microbiome and its interaction with the diet of an individual in order to predict an oral pH of the subject. In some embodiments, the prediction of an oral pH is based on the oral biological sample of the individual. In some embodiments, the diet information of the individual is collected through surveying the individual's lifestyle choices. In some embodiments, the diet information of the individual is collected through the residual diet signal within the individual's test results.

In some embodiments, the microbial, diet, and pH modeling engine, of the methods disclosed herein, is used to generate a recommendation regarding prebiotics, probiotics, or other diet components. In some embodiments, the recommended prebiotics, probiotics, or other diet components shift the oral cavity pH of the individual. In some embodiments, the methods disclosed herein make a recommendation regarding diet modifications, food supplements, probiotics, and prebiotics to modulate oral pH and promote a healthy oral microbiome. Poor oral health and cavity formation is associated with acidic pH. In some embodiments, the systematic, unbiased microbial characterization approach within the context of diet as disclosed herein, enables alternative solutions to the management of oral health. In some embodiments, the methods disclosed herein further identify food, nutrition, or diet components that exacerbate oral health through changing the pH of the oral cavity. In some embodiments, the methods disclosed herein identify food, nutrition, or diet components that improve oral health through changing the pH of the oral cavity

In some embodiments, the calculated abundance is used to recommend diet modifications and food supplements to change oral pH and promote a healthy oral microbiome. In some embodiments, the method further comprises generating a recommendation about certain diet options and other companion food supplements that shift the oral pH. In some embodiments, the shift in the oral pH promotes the growth of a microorganism population in the oral cavity. In some embodiments, the shift in the oral pH promotes the depletion of a microorganism population in the oral cavity. In some embodiments, the shift in the oral pH minimizes the risk of an oral and/or gum disease.

In some embodiments, the methods further comprise receiving a survey datum from the individual, the survey datum being coincident with the oral biological sample. In some embodiments, the machine learning algorithm further converts the survey datum into a quantitative score. In some embodiments, the survey datum is used to continually retrain the machine learning algorithm to adjust the cutoff abundance value. In some embodiments, the calculated abundance is associated with the survey datum. In some embodiments, the survey datum comprises dietary information, medical history, medical records, lifestyle information, or any combinations thereof, of the individual. In some embodiments, the lifestyle information comprises information regarding alcohol use, tobacco use, dental hygiene habits, or any combination thereof, of the individual. In some embodiments, the methods disclosed herein comprise longitudinal measurements of the oral microbiome within the context of relevant survey datum (e.g., lifestyle information) collected from the subject. In some embodiments, the methods disclosed herein use patient metadata. In some embodiments, the survey datum comprises patient metadata. In some embodiments, the patient metadata includes but is not limited to gender, age, occupation, ethnicity, number of cavities, history of oral or gum diseases, history of gum bleeding, history of gum recession, history of antibiotic usage, history of any medication usage, brushing routine, type of toothpaste used, usage of mouthwash, flossing routine, dietary information, vitamins, nutritional supplements, allergies, history of health conditions, smoking habits, and/or any other lifestyle information.

In some embodiments, the machine learning algorithm compares and correlates the calculated abundance to a gene expression profile of at least one gene in the oral biological sample. In some embodiments, the machine learning algorithm further compares the gene expression profile of the at least one gene in the oral biological sample to a reference gene expression profile of the at least one gene. In some embodiments, the at least one gene is differentially expressed in individuals who have an oral and/or gum disease as compared to individuals who do not have an oral and/or gum disease. In some embodiments, the reference gene expression profile is an average expression profile of the at least one gene in an oral biological sample of individuals who do not have an oral and/or gum disease. In some embodiments, the at least one gene is selected from MMP 10, MMP14, MMP16, MPPED2, ACTN2, VDR, FccRIIA, IL1-alpha, and IL1-beta.

Further disclosed herein, in certain embodiments, are methods of monitoring a progression of an oral and/or gum disease in an individual in need thereof.

In some embodiments, the methods comprise a) receiving a first plurality of sequence reads from a first oral biological sample of the individual and a second plurality of sequence read from a second oral biological sample of the individual. In some embodiments, the first oral biological sample and the second oral biological sample are a saliva sample, a biofilm sample, a dental tissue sample, a dental plaque sample, a tartar sample, a dental calculus sample, or any combination thereof.

In some embodiments, the first plurality of sequence reads and the second plurality of sequence reads corresponding to one or more nucleic acid molecules from at least one food source. In some embodiments, the first oral biological sample corresponds to a first collection time point and the second oral biological sample corresponds to a second collection time point. In some embodiments, the first oral biological sample and the second oral biological sample are collected from: a supragingival tissue, a subgingival tissue, a tongue, a buccal mucosa, a soft palate, a hard palate, a floor of a mouth, or any other anatomical location of the oral cavity of the individual. In some embodiments, the first oral biological sample and the second oral biological sample are a saliva sample, a biofilm sample, a dental tissue sample, a dental plaque sample, a tartar sample, a dental calculus sample, or any combination thereof.

In some embodiments, the first collection time point occurs one hour, six hours, twelve hours, twenty four hours, forty eight hours, seventy two hours, one week, four weeks, three months, six months, or twelve months prior to the second collection time point. In some embodiments, the first collection time point occurs about at least 1 hour to about 168 hours or more prior to the second collection time point. In some embodiments, the first collection time point occurs at least about 1 hour prior to the second collection time point. In some embodiments, the first collection time point occurs at most about 168 hours prior to the second collection time point. In some embodiments, the first collection time point occurs about 1 hour to about 2 hours, about 1 hour to about 4 hours, about 1 hour to about 6 hours, about 1 hour to about 12 hours, about 1 hour to about 24 hours, about 1 hour to about 48 hours, about 1 hour to about 72 hours, about 1 hour to about 96 hours, about 1 hour to about 120 hours, about 1 hour to about 144 hours, about 1 hour to about 168 hours, about 2 hours to about 4 hours, about 2 hours to about 6 hours, about 2 hours to about 12 hours, about 2 hours to about 24 hours, about 2 hours to about 48 hours, about 2 hours to about 72 hours, about 2 hours to about 96 hours, about 2 hours to about 120 hours, about 2 hours to about 144 hours, about 2 hours to about 168 hours, about 4 hours to about 6 hours, about 4 hours to about 12 hours, about 4 hours to about 24 hours, about 4 hours to about 48 hours, about 4 hours to about 72 hours, about 4 hours to about 96 hours, about 4 hours to about 120 hours, about 4 hours to about 144 hours, about 4 hours to about 168 hours, about 6 hours to about 12 hours, about 6 hours to about 24 hours, about 6 hours to about 48 hours, about 6 hours to about 72 hours, about 6 hours to about 96 hours, about 6 hours to about 120 hours, about 6 hours to about 144 hours, about 6 hours to about 168 hours, about 12 hours to about 24 hours, about 12 hours to about 48 hours, about 12 hours to about 72 hours, about 12 hours to about 96 hours, about 12 hours to about 120 hours, about 12 hours to about 144 hours, about 12 hours to about 168 hours, about 24 hours to about 48 hours, about 24 hours to about 72 hours, about 24 hours to about 96 hours, about 24 hours to about 120 hours, about 24 hours to about 144 hours, about 24 hours to about 168 hours, about 48 hours to about 72 hours, about 48 hours to about 96 hours, about 48 hours to about 120 hours, about 48 hours to about 144 hours, about 48 hours to about 168 hours, about 72 hours to about 96 hours, about 72 hours to about 120 hours, about 72 hours to about 144 hours, about 72 hours to about 168 hours, about 96 hours to about 120 hours, about 96 hours to about 144 hours, about 96 hours to about 168 hours, about 120 hours to about 144 hours, about 120 hours to about 168 hours, or about 144 hours to about 168 hours prior to the second collection time point. In some embodiments, the first collection time point occurs about 1 hour, about 2 hours, about 4 hours, about 6 hours, about 12 hours, about 24 hours, about 48 hours, about 72 hours, about 96 hours, about 120 hours, about 144 hours, or about 168 hours prior to the second collection time point.

In some embodiments, the individual is administered an oral and/or gum disease treatment after the first oral biological sample is collected. In some embodiments, the treatment is a medical treatment. In some embodiments, the medical treatment is administration of an antibiotic. In some embodiments, the treatment is a change in lifestyle. In some embodiments, the change in lifestyle is a brushing routine, a type of toothpaste, a usage of mouthwash, and/or a flossing routine. In some embodiments, the oral and/or gum disease treatment is a dental cleaning, a periodontal surgery, a gingival surgery, an antibiotic, a mouth wash, a fluoride treatment, a preventive restoration, an endodontic treatment, radiotherapy, chemotherapy, a biopsy, or any combination thereof. In some embodiments, the periodontal surgery and/or the gingival surgery is a gingivinoplasty, gingivectomy, flap surgery, mucogingival surgery, guided oral tissue regeneration, a synthetic bone graft surgery, a scaling and tooth planing procedure, a pocket reduction surgery, a native bone graft surgery, a soft tissue graft surgery, a bone surgery, or any combination thereof. In some embodiments, the antibiotic is an oral antibiotic or a systemic antibiotic. In some embodiments, the antibiotic is administered topically, systemically, orally, intravenously, or any combination thereof. In some embodiments, the antibiotic is penicillin, ampicillin, carbapenem, fluoroquinolone, cephalosporin, tetracycline, erythromycin, methicillin, gentamicin, vancomycin, imipenem, ceftazidime, levofloxacin, linezolid, daptomycin, ceftaroline, clindamycin, fluconazole, or ciprofloxacin. In some embodiments, the fluoride treatment is topical or systemic. In some embodiments, the treatment comprises the use of pit and/or fissure sealants. In some embodiments, the preventive restoration comprises a root canal treatment, scaling and/or polishing of teeth, filling a cavity with a dental metal amalgam and/or a resin composite, or any combination thereof. In some embodiments, the treatment is an indirect restoration. In some embodiments, the indirect restoration comprises a full crown restoration, an inlay restoration, an intracoronal restoration, an onlay restoration, a dental veneer procedure, or any combination thereof.

In some embodiments, the treatment is anon-medical intervention. In some embodiments, the non-medical intervention is nutrition and diet changes, oral health education, proper methods of maintaining oral hygiene, ceasing oral abusive habits (e.g., tobacco smoking and/or tobacco chewing), removal of irritants from oral cavity, undergoing regular oral check-ups, or any combination thereof. In some embodiments, proper methods of maintaining oral hygiene comprise use of fluoride toothpaste and toothbrush, use of dental floss, use of interdental brushes, antiseptic mouth washes, or any combination thereof.

In some embodiments, the methods disclosed herein measure a response to an oral and/or gum disease treatment or to an intervention strategy. In some embodiments, the calculated abundance is used to measure response to oral or gum disease treatment. In some embodiments, a response to the oral and/or gum disease treatment is measured based on the indicator of the progression of the oral and/or gum disease. In some embodiments, the methods measure the amount of a response to a treatment. In some embodiments, the methods quantify a response to a treatment.

In some embodiments, the methods comprise b) taxonomically classifying the first plurality of sequence reads and the second plurality of sequence reads to identify the at least one food source. In some embodiments, the methods comprise c) quantifying the one or more nucleic acid molecules from the identified at least one food source. In some embodiments, the methods comprise generating a first calculated abundance and a second calculated abundance of the identified at least one food source.

In some embodiments, the methods comprise d) applying a machine learning algorithm. In some embodiments, the machine learning algorithm compares the first calculated abundance and the second calculated abundance to a cutoff abundance value associated with a healthy oral biological sample or with a diseased oral biological sample. In some embodiments, the machine learning algorithm calculates a first correlation value between the first calculated abundance and the cutoff abundance value. In some embodiments, the machine learning algorithm calculates a second correlation value between the second calculated abundance and the cutoff abundance value. In some embodiments, the machine learning algorithm compares the first correlation value to the second correlation value. In some embodiments, the machine learning algorithm provides an indicator of the progression of the oral and/or gum disease in the individual based on the comparison between the first correlation value and the second correlation value. In some embodiments, the first calculated abundance and the second calculated abundance are used to continually retrain the machine learning algorithm to adjust the cutoff abundance value.

In some embodiments, identified information and/or recommendations based on the identified information can be provided to an application (such as a web browser) installed on a handheld or other type of personal computing device. For example, a dentist may employ such an application to obtain identified information and/or recommendation for a patient.

Example Collection Methodology

In some embodiments, the above described samples are collected by dentists across various fields of dentistry (periodontists, orthodontists, endodontists, general dentists, and biological dentists). Areas in which samples can be collected include, but are not limited to, extracted tooth, tongue, cheek, plaque, nerve, gingival suclus, root canal cavity, active gingivitis, nasal cavity, healthy gum, and healthy pockets between tooth and gum.

In some embodiments, samples can be collected using a swab method, a paper point method, or a dental scaling tools method. In some embodiments, the swab method is recommended for taking samples from the tongue, gum, and saliva. For example, after sample collection, the swab is placed inside the collection tube. In some embodiments, the swab has a notch in the middle. After the sample is placed inside the tube, a dentist (or technician) breaks the swab at the notch and close the cap.

In some embodiments, the paper point method is recommended for taking samples from the root canal cavity or infected tooth/gum. In some embodiments, paper point method includes dropping a paper point inside a sample collection tube after a sample is collected. Dentists or technicians can use any sterile paper point.

In some embodiments, the dental scaling tools method is recommended for taking samples from the pockets between the gum and tooth. Also, removed tartar or any specimen removed during the root canal procedure can be collected using this method. In some embodiments, the dental scaling tools method includes placing a specimen on a swab and then placing the swab in a sample collection tube.

In some embodiments, after a sample is placed inside of a collection tube, the tube is capped. The sample collection date, the name of patient, and the method used to collect the sample can be has written on the tube. In some embodiments, the specimen tube is placed inside a specimen bag. The specimen may then be shipped to the collection location using a shipment bag.

In some embodiments, the sample is prepare for sequencing after it is received. In some embodiments, the received sample is sequenced and the data reviewed and/or used to train a machine learning model.

In some embodiments, to keep patient confidentiality, each dentist can assign a number as, for example, metadata, to each patient that can trace back to the actual patient by only the patient's particular dentist. In some embodiments, the numbers within the metadata are matched to the respective sample. In some embodiments, a barcode based sample collection method can be employed to assign a unique identifier to each collected sample data.

Computer-Implemented Systems

Additionally disclosed herein, in certain embodiments, are systems for analyzing an oral biological sample from an individual. In some embodiments, the oral biological sample comprises one or more nucleic acid molecules. In some embodiments, the one or more nucleic acid molecules are from at least one food source. In some embodiments, the food source is a plant source, an animal source, or an edible fungal source. In some embodiments, the plant source is a plant from the kingdom of Thallophyta, Pteridophyta, or Spermatophyta. In some embodiments, the animal source is an invertebrate animal source or a vertebrate animal source. In some embodiments, the edible fungal source is a fungus from the Ascomycota phylum, Basidiomycota phylum, Blastocladiomycota phylum, Chytridiomycota phylum, Glomeromycota phylum, Microsporidia phylum, or Neocallimastigomycota phylum. In some embodiments, the oral biological sample comprises one or more nucleic acid molecules from a microorganism. In some embodiments, the microorganism is a bacterium, a virus, or a fungus.

In some embodiments, the system comprises a digital processing device. In some embodiments, the digital processing device comprises at least one processor, an operating system configured to perform executable instructions, a memory, and a computer program including the instructions executable by the digital processing device. In some embodiments, the instructions comprise receiving a plurality of sequence reads corresponding to the one or more nucleic acid molecules from the at least one food source. In some embodiments, the instructions comprise taxonomically classifying the plurality of sequence reads to identify the at least one food source. In some embodiments, the instructions comprise quantifying the one or more nucleic acid molecules from the identified at least one food source, thereby generating a calculated abundance of the identified at least one food source. In some embodiments, the calculated abundance is used to continually retrain the machine learning algorithm to adjust the cutoff abundance value.

In some embodiments, the instructions further comprise receiving a plurality of sequence reads corresponding to the one or more nucleic acid molecules from the microorganism. In some embodiments, the instructions comprise taxonomically classifying the plurality of sequence reads to identify the at least one microorganism. In some embodiments, the instructions comprise quantifying the one or more nucleic acid molecules from the identified at least one microorganism, thereby generating a calculated abundance of the identified at least one microorganism.

In some embodiments, the instructions comprise applying a machine learning algorithm. In some embodiments, the machine learning algorithm compares the calculated abundance to a cutoff abundance value associated with a healthy oral biological sample or with a diseased oral biological sample. In some embodiments, the machine learning algorithm calculates a correlation value between the calculated abundance and the cutoff abundance value. In some embodiments, the calculated abundance of the at least one food source and the calculated abundance of the microorganism are used to estimate a pH value of the oral cavity of the individual. In some embodiments, the machine learning algorithm further compares the calculated abundance to the pH value.

In some embodiments, the machine learning algorithm provides an indicator of an oral disease in the oral biological sample based on the correlation value. In some embodiments, the machine learning algorithm provides an indicator of a stage of a disease. In some embodiments, the machine learning algorithm provides an indicator of a risk of a disease. In some embodiments, the disease is an oral and/or gum disease.

In some embodiments, the machine learning algorithm provides an indicator of a therapeutic solution. In some embodiments, the therapeutic solution comprises a therapeutic agent or a dietary supplement. In some embodiments, the therapeutic agent is an over the counter therapeutic agent. In some embodiments, the dietary supplement is a probiotic. In some embodiments, the dietary supplement is a vitamin, a mineral, a fiber, a fatty acid, an amino acid, a protein, a natural product, or any combination thereof. In some embodiments, the natural product comprises intact samples or extracts from plants, animals, fungi or lichens, algae, or any combination thereof.

In some embodiments, the instructions further comprise receiving a survey datum from the individual, the survey datum being coincident with the oral biological sample. In some embodiments, the machine learning algorithm further converts the survey datum into a quantitative score. In some embodiments, the survey datum is used to continually retrain the machine learning algorithm to adjust the cutoff abundance value. In some embodiments, the calculated abundance is associated with the survey datum. In some embodiments, the survey datum comprises dietary information, medical history, medical records, lifestyle information, or any combinations thereof, of the individual. In some embodiments, the lifestyle information comprises information regarding alcohol use, tobacco use, dental hygiene habits, or any combination thereof, of the individual. In some embodiments, the machine learning algorithm compares and correlates the calculated abundance to a gene expression profile of at least one gene in the oral biological sample. In some embodiments, the machine learning algorithm further compares the gene expression profile of the at least one gene in the oral biological sample to a reference gene expression profile of the at least one gene. In some embodiments, the at least one gene is differentially expressed in individuals who have an oral disease as compared to individuals who do not have an oral disease. In some embodiments, the reference gene expression profile is an average expression profile of the at least one gene in an oral biological sample of individuals who do not have an oral disease. In some embodiments, the at least one gene is selected from MMP 10, MMP14, MMP16, MPPED2, ACTN2, VDR, FccRIIA, IL1-alpha, and IL1-beta.

Machine Learning

In some embodiments, machine learning algorithms are employed to aid in determining a provide an indicator of an oral and/or gum disease in the individual based on the correlation value. Examples of machine learning algorithms may include a support vector machine (SVM), a naïve Bayes classification, a random forest, a neural network, deep learning, or other supervised learning algorithm or unsupervised learning algorithm for classification and regression. The machine learning algorithms may be trained using one or more training datasets. In some embodiments, the machine learning algorithm utilizes regression modelling, wherein relationships between predictor variables and dependent variables are determined and weighted.

Algorithms

In some embodiments, a machine learning model comprises one or more machine learning algorithms, such as a multi-variate linear regression model. In some embodiments, the machine learning model comprises one or more machine learning algorithms that mimic a system (e.g., food and/or bacteria having an effect on the health or progression of a disease in a patient). In some embodiments, the machine learning algorithm comprises a classifier. In some embodiments, the machine learning model classifies oral biological sample data. In some embodiments, the machine learning model comprises a regressor. In some embodiments, the machine learning model makes a prediction based on a regression function.

In some embodiments, the machine learning algorithm is an unsupervised machine learning algorithm. In some embodiments, the machine learning algorithm is a supervised machine learning algorithm. In some embodiments, the system comprises both an unsupervised machine learning algorithm and a supervised machine learning algorithm. In some embodiments, the machine learning algorithm is a random forest model, a tSNE model an artificial neural network model a decision tree model, a kNN model, a PCA model, a TCA classifier, a deep neural network model, a support vector machine model, or a linear classification model. In some embodiments, the machine learning algorithm is an ensemble of multiple similar or different models. In some embodiments, the machine learning algorithm is a combination of one or more of the following algorithms: a random forest model, a tSNE model an artificial neural network model a decision tree model, a kNN model, a PCA model, a TCA classifier, a deep neural network model, a support vector machine model, and/or a linear classification model.

In some embodiments, the systems disclosed herein use pattern recognition and unsupervised clustering algorithms to identify most occurring communities of food sources and/or microorganisms in the oral biological sample. In some embodiments, the system identifies the most prevalent components of oral health. In some embodiments, the machine learning algorithm is a tSNE model. In some embodiments, the tSNE model is a visualization tool. In some embodiments, the tSNE model helps classify the collected data (e.g., the plurality of sequences). In some embodiments, the tSNE model is used to visualize the collected data. In some embodiments, a combination of the tSNE model (used to visualize the collected data) and one or more additional algorithms (used to classify the collected data) is used in the methods and systems disclosed herein. In some embodiments, the tSNE model is used in combination with an additional algorithm that classifies the collected data. In some embodiments, the methods and systems disclosed herein do not use any visualization tools. In some embodiments, the methods and systems disclosed herein do not use the tSNE model. In some embodiments, the methods and systems disclosed herein rely only on manual classification of the collected data.

In some embodiments, the machine learning algorithm is a PCA model. In some embodiments, the machine learning algorithm is a random forest model. In some embodiments, the machine learning algorithm is an artificial neural network model. In some embodiments, the machine learning algorithm is a deep neural network model. In some embodiments, the machine learning algorithm is a recurrent neural network model. In some embodiments, the machine learning algorithm is a decision tree model. In some embodiments, the machine learning algorithm is a kNN model. In some embodiments, the machine learning algorithm is a TCA classifier. In some embodiments, the machine learning algorithm is a support vector machine model. In some embodiments, the machine learning algorithm is a linear classification model. In some embodiments, the machine learning algorithm is used to segregate essential components in each of these microbial communities.

In some embodiments, the systems disclosed herein use clustering algorithms and metabolic modeling to identify functional elements within microbial communities. In some embodiments, the functional elements are essential to establish and maintain a healthy oral microbiome. In some embodiments, the system comprises identifying the biochemical pathways and functions that dictate establishing a healthy microbial community.

In some embodiments, pattern recognition machine learning algorithms are used for characterization and clustering of communities of microorganisms in healthy or disease cohorts that underlie oral health. In some embodiments, the combination of machine learning algorithms and metabolic modeling are used to identify effective functional elements in the microbial communities. In some embodiments, machine learning and other analytical algorithms are used to compare differential microbial communities and their effective functional elements. In some embodiments, machine learning algorithms identify which functional elements, small molecules, biochemical pathways, or active agents are essential in growth or elimination of undesired microbial communities.

In some embodiments, the systems disclosed herein use machine learning or other unsupervised machine learning algorithms for unbiased classification of individual or network level biological elements that characterize oral health or disease. In some embodiments, the systems disclosed herein use combination of untargeted characterization methodology with machine learning or other unsupervised machine learning algorithms to comprehensively characterize the individual or network level biological elements that characterize oral health or disease.

Computer Control Systems

The present disclosure provides computer control systems that are programmed to implement methods of the disclosure. FIG. 1 shows a computer system 101 that is programmed or otherwise configured to, for example, analyze an oral biological sample of an individual. In some embodiments, the computer system 101 regulates various aspects of methods, and systems of the present disclosure, such as, for example, predicting an oral and/or gum disease and monitoring the progression of an oral and/or gum disease in an individual. In some embodiments, the computer system 101 is an electronic device of a user or a computer system that is remotely located with respect to the electronic device. In some embodiments, the electronic device is a mobile electronic device.

In some embodiments, the computer system 101 includes a central processing unit (CPU, also “processor” and “computer processor” herein) 105, which can be a single core or multi core processor, or a plurality of processors for parallel processing. In some embodiments, the computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. In some embodiments, the memory 110, storage unit 115, interface 120 and peripheral devices 125 are in communication with the CPU 105 through a communication bus (solid lines), such as a motherboard. In some embodiments, the storage unit 115 is a data storage unit (or data repository) for storing data. In some embodiments, the computer system 101 is operatively coupled to a computer network (“network”) 130 with the aid of the communication interface 120. In some embodiments, the network 130 is the Internet, an internet and/or extranet, or an intranet and/or extranet that is in communication with the Internet. In some embodiments, the network 130 is a telecommunication and/or data network. In some embodiments, the network 130 includes one or more computer servers, which enable distributed computing, such as cloud computing. In some embodiments, the network 130, with the aid of the computer system 101, implements a peer-to-peer network, which enables devices coupled to the computer system 101 to behave as a client or a server.

In some embodiments, the CPU 105 executes a sequence of machine-readable instructions, which are embodied in a program or software. In some embodiments, the instructions are stored in a memory location, such as the memory 110. In some embodiments, the instructions are directed to the CPU 105, which subsequently program or otherwise configure the CPU 105 to implement methods of the present disclosure. Non-limiting examples of operations performed by the CPU 105 include fetch, decode, execute, and writeback.

In some embodiments, the CPU 105 is part of a circuit, such as an integrated circuit. In some embodiments, one or more other components of the system 101 are included in the circuit. In some cases, the circuit is an application specific integrated circuit (ASIC).

In some embodiments, the storage unit 115 stores files, such as drivers, libraries, and saved programs. In some embodiments, the storage unit 115 stores user data, e.g., user preferences and user programs. In some embodiments, the computer system 101 includes one or more additional data storage units that are external to the computer system 101, such as located on a remote server that is in communication with the computer system 101 through an intranet or the Internet.

In some embodiments, the computer system 101 communicates with one or more remote computer systems through the network 130. For instance, in some embodiments, the computer system 101 communicates with a remote computer system of a user (e.g., a personal computer). Non-limiting examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. In some embodiments, the user accesses the computer system 101 via the network 130.

In some embodiments, methods as described herein are implemented by way of machine (e.g., computer processor) executable code stored on an electronic storage location of the computer system 101, such as, for example, on the memory 110 or electronic storage unit 115. In some embodiments, the machine executable or machine readable code is provided in the form of software. In some embodiments, during use, the code is executed by the processor 105. In some embodiments, the code is retrieved from the storage unit 915 and stored on the memory 110 for ready access by the processor 105. In some embodiments, the electronic storage unit 115 is precluded, and machine-executable instructions are stored on memory 110.

In some embodiments, the code is pre-compiled and configured for use with a machine having a processor adapted to execute the code, or is compiled during runtime. In some embodiments, the code is supplied in a programming language that is selected to enable the code to execute in a pre-compiled or as-compiled fashion.

In some embodiments, aspects of the systems and methods provided herein, such as the computer system 101, are embodied in programming. In some embodiments, various aspects of the technology are thought of as “products” or “articles of manufacture” typically in the form of machine (or processor) executable code and/or associated data that is carried on or embodied in a type of machine readable medium. In some embodiments, machine-executable code is stored on an electronic storage unit, such as memory (e.g., read-only memory, random-access memory, flash memory) or a hard disk. In some embodiments, “storage” type media includes any or all of the tangible memory of the computers, processors or the like, or associated modules thereof, such as various semiconductor memories, tape drives, disk drives and the like, which provide non-transitory storage at any time for the software programming. In some embodiments, all or portions of the software are, at times, communicated through the Internet or various other telecommunication networks. In some embodiments, such communications, for example, enable loading of the software from one computer or processor into another, for example, from a management server or host computer into the computer platform of an application server. Thus, another type of media that bears the software elements, in some embodiments, includes optical, electrical and electromagnetic waves, such as used across physical interfaces between local devices, through wired and optical landline networks and over various air-links. In some embodiments, the physical elements that carry such waves, such as wired or wireless links, optical links or the like, are also considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, in some embodiments, takes many forms, including but not limited to, a tangible storage medium, a carrier wave medium, or a physical transmission medium. In some embodiments, non-volatile storage media include, for example, optical or magnetic disks, such as any of the storage devices in any computer(s) or the like, such as are used to implement the databases, etc. shown in the drawings. In some embodiments, volatile storage media include dynamic memory, such as main memory of such a computer platform. In some embodiments, tangible transmission media include coaxial cables; copper wire and fiber optics, including the wires that comprise a bus within a computer system. In some embodiments, carrier-wave transmission media take the form of electric or electromagnetic signals, or acoustic or light waves such as those generated during radio frequency (RF) and infrared (IR) data communications. In some embodiments, common forms of computer-readable media therefore include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. In some embodiments, many of these forms of computer readable media are involved in carrying one or more sequences of one or more instructions to a processor for execution.

In some embodiments, the computer system 101 includes or is in communication with an electronic display 135 that comprises a user interface (UI) 140 for providing, for example, the indicator of an oral and/or gum disease. Examples of UI's include, without limitation, a graphical user interface (GUI) and web-based user interface.

In some embodiments, methods and systems of the present disclosure are implemented by way of one or more algorithms. In some embodiments, an algorithm is implemented by way of software upon execution by the central processing unit 105. In some embodiments, the algorithm, for example, identifies a food source and/or a microorganism source in the oral biological sample of the individual, predicts an oral and/or gum disease, and/or monitors the progression of an oral and/or gum disease in an individual.

### ADDITIONAL EMBODIMENTS

In some embodiments, the methods and systems disclosed herein use multiple techniques to identify oral microbiome flora and other biomarkers derived from characterization of an individual's oral health or hygiene. In some embodiments, the methods and systems disclosed herein predict the onset of oral or gum diseases, or any other deviation from oral well-being. In some embodiments, the methods and systems disclosed herein monitor progression of oral or gum disease. In some embodiments, the methods and systems disclosed herein make a recommendation regarding an effective treatment for specific oral or gum diseases. In some embodiments, the methods and systems disclosed herein identify bacterial genera, species, or strains that exacerbate oral health. In some embodiments, the methods and systems disclosed herein identify fungal genera, species, or strains that exacerbate oral health. In some embodiments, the methods and systems disclosed herein identify viruses and bacteriophages that exacerbate oral health. In some embodiments, the methods and systems disclosed herein identify bacterial genera, species, or strains that improve oral health. In some embodiments, the methods and systems disclosed herein identify fungal genera, species, or strains that improve oral health. In some embodiments, the methods and systems disclosed herein identify viruses and bacteriophages that improve oral health. In some embodiments, the methods and systems disclosed herein identify food, nutrition, or diet components that exacerbate oral health through enhancement of undesirable bacterial genera, species, or strains. In some embodiments, the methods and systems disclosed herein identify food, nutrition, or diet components that exacerbate oral health through enhancement of undesirable fungal genera, species, or strains. In some embodiments, the methods and systems disclosed herein identify food, nutrition, or diet components that exacerbate oral health through enhancement of undesirable viruses and bacteriophages. In some embodiments, the methods and systems disclosed herein identify food, nutrition, or diet components that improve oral health through enhancement of beneficial bacterial genera, species, or strains. In some embodiments, the methods and systems disclosed herein identify food, nutrition, or diet components that improve oral health through enhancement of beneficial fungal genera, species, or strains. In some embodiments, the methods and systems disclosed herein identify food, nutrition, or diet components that improve oral health through enhancement of beneficial viruses and bacteriophages. In some embodiments, the methods and systems disclosed herein makes a recommendation regarding an oral health shifting generic solution (mouth rinse, mouthwash, toothpaste) to treat specific oral or gum diseases. In some embodiments, the methods and systems disclosed herein makes a recommendation regarding a cleansing or wash solution that can help clean the baseline and boost the efficacy of the administered supplement, prebiotics, or probiotics. In some embodiments, the methods and systems disclosed herein makes a recommendation regarding a combination of a cleansing or wash solution with an oral health shifting solution as part of a two-phase solution that can help clean the baseline and boost the efficacy of the administered supplement, prebiotics, probiotics, or any other oral health shifting solution.

In some embodiments, the methods and systems disclosed herein use characterization of networks or cohorts of oral microbiota or microbiome community signatures. In some embodiments, the methods and systems disclosed herein identify classes of microbial communities that most prevalently found in oral microbiome. In some embodiments, the methods and systems disclosed herein identify essential elements of a healthy oral microbiome. In some embodiments, the methods and systems disclosed herein identify essential effective functional elements for a healthy oral microbiome. In some embodiments, the methods and systems disclosed herein identify food elements, diet components, or supplements targeted towards improvement of specific oral or gum diseases. In some embodiments, the methods and systems disclosed herein use an un-targeted methodology for culture independent characterization of microbial species or communities that signify oral health or disease.

In some embodiments, the collected data is used to predict an oral or gum disease. In some embodiments, the methods and systems disclosed herein comprise collecting and analyzing an oral biological sample at various time points. In some embodiments, at each sample collection time point, the method and system calculate the abundance of each bacterial, fungal, and viral genera, species, or strain. In some embodiments, at each sample collection time point, the method and system compare the calculated abundance against a signature data model. In some embodiments, the signature data model is a database of healthy and dysbiotic microbiome communities. Oral and gum diseases have specific bacterial, fungal species associated to them.

In some embodiments, the method and system predict the existence of different oral or gum diseases by measuring the abundance of specific bacteria, fungi, and viruses.

In some embodiments, the machine learning enabled network level analysis allows higher order interactions between different members of the bacterial communities to be captured as diagnostic measures of oral health. In some embodiments, the method and system detect changes and trends in the abundance of different bacteria, fungi, and viruses by longitudinal measurements of the oral microbiome flora and relevant lifestyle information. In some embodiments, the longitudinal studies are used to determine baselines of microorganism populations. In some embodiments, the method and system recognize deviations from the baselines and use these deviations to predict an onset of associated oral or gum diseases.

In some embodiments, the history of oral health measures of an individual is evaluated within the context of their personalized baseline of microorganism populations. In some embodiments, concurrent evaluation of the history of oral health measures and the baseline of microorganism populations increases the accuracy of the assessment of the oral health of the individual.

In some embodiments, the collected data is used to monitor an oral or gum disease progression. In some embodiments, the method and system detect changes and trends in the abundance of different bacteria, fungi, and viruses and correlates the changes and trends to the progression of associated oral or gum diseases.

In some embodiments, the methods and systems disclosed herein comprise longitudinal measurements of the oral microbiome. In some embodiments, the method and system detect changes and trends in the abundance of different bacteria, fungi, and viruses.

In some embodiments, the collected data is used to suggest an effective treatment for a specific oral or gum disease. In some embodiments, the methods and systems disclosed herein identify and recommend treatment options for a specific oral or gum disease by analyzing the oral microbiome of the individual. In some embodiments, such treatment options include but are not limited to effective antibiotic treatment, proprietary biologics with desired function, and/or generic dental hygiene products. Non-limiting examples of the generic dental hygiene products include floss, toothpaste, and mouthwash.

In some embodiments, the method and system correlates the composition of the oral microbiome of different cohorts with a diet signal in any individual's test results, with their lifestyle diet information, or with both.

In some embodiments, the method and system identifies microbial communities associated with an oral or gum disease or overall oral health of the individual by measuring the oral microbiota and/or microbiome of the individual. In some embodiments, the method or system identifies and recommends supplements, diet elements, probiotics, and/or prebiotics that promote the health and well-being of the oral cavity of the individual.

In some embodiments, the collected data is used to identify classes of bacterial, fungal, and viral communities that occur most often in the oral microbiome. In some embodiments, the collected data is used to identify essential elements of a healthy microbiome. In some embodiments, the method and system comprise a holistic model including the metadata of the patient. In some embodiments, the holistic model assesses and predicts an individual's oral health. In some embodiments, the holistic model uses a combination of microbial community elements and the metadata of the patient (e.g., lifestyle choices) that together define the well-being of an individual's oral health.

In some embodiments, the collected data is used to identify essential effective functional elements for a healthy oral microbiome. In some embodiments, the collected data is used to identify food supplements or prebiotics for specific oral or gum diseases. In some embodiments, the functional elements, active agents, or small molecules are used to eliminate associated microorganism and/or promote growth of mutualistic or commensal microorganisms.

In some embodiments, administrating a combination of the supplement, prebiotic, probiotic, or other oral health-shifting agent with a cleansing solution or a wash solution increases the efficacy of the administered supplement, prebiotic, or probiotic. In some embodiments, the efficacy is increased by about 1%. In some embodiments, the efficacy is increased by about 5%. In some embodiments, the efficacy is increased by about 10%. In some embodiments, the efficacy is increased by about 15%. In some embodiments, the efficacy is increased by about 20%. In some embodiments, the efficacy is increased by about 30%. In some embodiments, the efficacy is increased by about 40%. In some embodiments, the efficacy is increased by about 50%. In some embodiments, the efficacy is increased by about 60%. In some embodiments, the efficacy is increased by about 70%. In some embodiments, the efficacy is increased by about 80%. In some embodiments, the efficacy is increased by about 90%. In some embodiments, the efficacy is increased by about 100%.

In some embodiments, the cleansing solution or the wash solution clean the baseline population of a microorganism in the oral cavity of the individual. In some embodiments, a cleansing or wash solution and an oral health-shifting agent are administered to the individual. In some embodiments, a method of treating an oral disease comprises administering the cleansing or wash solution to the individual as a first step and administering the oral health-shifting agent to the individual as a second step. In some embodiments, the cleansing or wash solution has targeted or generic antimicrobial activity. In some embodiments, the cleansing or wash solution is prescribed for routine usage. In some embodiments, upon usage of the cleansing or wash solution, the overall population of the microorganism is depleted within the oral cavity. In some embodiments, upon usage of the cleansing or wash solution, the subset of the population of the microorganism is depleted within the oral cavity. In some embodiments, given the accessibility of the oral cavity, such depletion will have a topical and non-systemic impact on the microbial communities within the oral cavity.

In some embodiments, after usage of the cleansing or wash solution, the supplement, prebiotic, or probiotic with anticipated oral health-shifting activity is used. Because of the attenuated baseline population of the microorganism within the original community, the oral health-shifting agent has an increased efficacy in terms of establishing the desired microbial community. In some embodiments, the efficacy is increased by about 1% compared to administering the oral health-shifting agent alone. In some embodiments, the efficacy is increased by about 5% compared to administering the oral health-shifting agent alone. In some embodiments, the efficacy is increased by about 10% compared to administering the oral health-shifting agent alone. In some embodiments, the efficacy is increased by about 15% compared to administering the oral health-shifting agent alone. In some embodiments, the efficacy is increased by about 20% compared to administering the oral health-shifting agent alone. In some embodiments, the efficacy is increased by about 30% compared to administering the oral health-shifting agent alone. In some embodiments, the efficacy is increased by about 40% compared to administering the oral health-shifting agent alone. In some embodiments, the efficacy is increased by about 50% compared to administering the oral health-shifting agent alone. In some embodiments, the efficacy is increased by about 60% compared to administering the oral health-shifting agent alone. In some embodiments, the efficacy is increased by about 70% compared to administering the oral health-shifting agent alone. In some embodiments, the efficacy is increased by about 80% compared to administering the oral health-shifting agent alone. In some embodiments, the efficacy is increased by about 90% compared to administering the oral health-shifting agent alone. In some embodiments, the efficacy is increased by about 100% compared to administering the oral health-shifting agent alone.

### EXAMPLES

### Example 1—Predicting an Oral and/or Gum Disease Based on a Food Source Abundance

A dentist collects an oral biological sample from a supragingival tissue in the oral cavity of a patient undergoing a regular prophylaxis appointment. The oral biological sample is sequenced using a Next Generation Sequencing technique yielding a plurality of sequence reads. The plurality of sequence reads is taxonomically classified in order to identify at least one food source in the oral biological sample. At least one food source is identified and it is further quantified in order to generated a calculated abundance of the at least one food source. A machine learning algorithm is further applied in order to compare the calculated abundance to a cutoff abundance value associated with a diseased oral biological sample obtained from an individual suffering from caries. The machine learning algorithm calculates a correlation value between the calculated abundance and the cutoff abundance value. Next, the machine learning algorithm provides an indicator of caries (e.g., a value representing the risk of caries) of the oral biological sample of the patient, based on the correlation value. The machine learning algorithm determines the risk of the patient developing at least one caries is about 70% to about 85%. The machine learning algorithm further detects elevated abundances of sugary food sources and a low pH in the oral biological sample of the patient. The machine learning algorithm recommends a decrease in sugary food intake as a preventative measure and as a way to increase pH levels to a normal range. The dentist receives the results (i.e., the indicator of caries and the suggested prophylactic recommendations) through, for example, an application (e.g., a web browser) installed on a handle held device or other type device and relays the information to the patient.

### Example 2—Predicting an Oral and/or Gum Disease Based on a Food Source Abundance, a Bacterial Abundance, and Metadata

A dentist collects an oral biological sample from a supragingival tissue in the oral cavity of a patient undergoing a regular prophylaxis appointment. The oral biological sample is sequenced using a Next Generation Sequencing technique yielding a plurality of sequence reads. The plurality of sequence reads is taxonomically classified in order to identify at least one food source in the oral biological sample. At least one food source is identified and it is further quantified in order to generated a calculated abundance of the at least one food source. Furthermore, the plurality of sequence reads is taxonomically classified in order to identify at least one bacterial source in the oral biological sample. At least one bacterial source is identified and it is further quantified in order to generated a calculated abundance of the at least one bacterial source.

A machine learning algorithm is further applied in order to compare the calculated abundances (i.e., food and bacterial calculated abundances) to a cutoff abundance value associated with a diseased oral biological sample obtained from an individual suffering from periodontitis. The machine learning algorithm calculates a first correlation value between the calculated abundance and the cutoff abundance value.

In addition, metadata of the patient including their age, dietary information, and history of oral and/or gum diseases is input into the machine learning algorithm. The machine learning algorithm is further applied in order to compare the metadata of the patient to cutoff values, for each metadata category, associated with a diseased oral biological sample obtained from an individual suffering from periodontitis. The machine learning algorithm calculates a second correlation value between the calculated abundance and the cutoff abundance value.

Next, the machine learning algorithm provides an indicator of periodontitis (e.g., a value representing the risk of periodontitis) of the oral biological sample of the patient, based on the first and second correlation values. The machine learning algorithm determines the risk of the patient developing periodontitis is about 50% to about 65%. The machine learning algorithm further detects elevated abundances of A. actinomycetemcomitans, P. gingivalis, P. intermedia, B. forsythus, C. rectus, E. nodatum, P. micros, S. intermedius and Treponema sp. and a low pH in the oral biological sample of the patient. The machine learning algorithm recommends a decrease in sugary food intake as a preventative measure and as a way to increase pH levels to a normal range. Additionally, the machine learning algorithm recommends regular use of an antiseptic mouthwash as a preventative measure. The dentist receives the results (i.e., the indicator of caries and the suggested prophylactic recommendations) through, for example, an application (e.g., a web browser) installed on a handle held device or other type device and relays the information to the patient. While preferred embodiments of the present invention have been shown and described herein, it will be obvious to those skilled in the art that such embodiments are provided by way of example only. It is not intended that the invention be limited by the specific examples provided within the specification.

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented system for providing a recommendation for an individual regarding oral health based on an analysis of an oral biological sample from the individual, the system comprising:
a computing device comprising a user interface;
at least one processor, and
a computer-readable storage device coupled to the at least one processor and having instructions stored thereon which, when executed by the at least one processor, cause the at least one processor to perform operations comprising:
receiving a plurality of sequence reads of the oral biological sample collected from the individual, wherein each of the sequence reads corresponding to one or more nucleic acid molecules from at least one food source or at least one microorganism;
determining an abundance of the at least one food source or the at least one microorganism by:
taxonomically classifying the sequence reads to identify the at least one food source or the at least one microorganism; and
quantifying the one or more nucleic acid molecules from the identified at least one food source or the at least one microorganism;
processing the abundance of the at least one food source or the at least one microorganism through a machine learning algorithm to determine an indicator of an oral or gum disease in the individual, the machine learning algorithm having been trained using a plurality previously received sequence reads of oral biological samples collected from other individuals; and
providing, to the user interface, the recommendation for the individual regarding oral health based on the indicator of the oral or gum disease in the individual.

2. The system of claim 1, wherein the indicator of an oral or gum disease in the individual is determined based on a correlation value between the abundance of the at least one food source or the at least one microorganism and a cutoff abundance value.

3. The system of claim 2, wherein the operations comprise:
retraining the machine learning algorithm with the abundance of the at least one food source or the at least one microorganism to adjust the cutoff abundance value.

4. The system of claim 2, wherein the cutoff abundance value is associated with a healthy oral biological sample, a diseased oral biological sample, or a risk or probability of disease.

5. The system of claim 4, wherein the oral biological samples collected from the other individuals comprise the healthy oral biological sample and the diseased oral biological sample.

6. The system of claim 2, wherein the operations comprise:
receiving a survey datum from the individual, the survey datum being coincident with the oral biological sample
processing the survey datum through the machine learning algorithm to determine a quantitative score.

7. The system of claim 6, wherein the operations comprise:
retraining the machine learning algorithm with survey datum to adjust the cutoff abundance value, wherein the abundance is associated with the survey datum.

8. The system of claim 7, wherein the survey datum comprises dietary information, medical history, medical records, lifestyle information, or any combinations thereof, of the individual.

9. The system of claim 1, wherein the operations comprise:
receiving a gene expression profile of at least one gene in the oral biological sample collected from the individual; and
processing the gene expression profile through the machine learning algorithm to determine the indicator of an oral or gum disease in the individual.

10. The system of claim 9, wherein the indicator of an oral or gum disease in the individual is determined based on a correlation value between the gene expression profile and a reference gene expression profile of the at least one gene, wherein the reference gene expression profile is determined based on the expression profiles of the at least one gene in oral biological samples of other individuals who do not have an oral and/or gum disease.

11. The system of claim 9, wherein the at least one gene is differentially expressed in individuals who have an oral and/or gum disease as compared to individuals who do not have an oral and/or gum disease.

12. The system of claim 9, wherein the at least one gene is selected from Matrix MetalloProtease-10 (MMP10), Matrix MetalloProtease-14 (MMP14), Matrix MetalloProtease-16 (MMP16), Metallophosphoesterase Domain Containing 2 (MPPED2), Actinin Alpha 2 (ACTN2), vitamin D receptor (VDR), FccRIIA, Interleukin1-alpha (IL1-alpha), and Interleukin1-beta (IL1-beta).

13. The system of claim 1, wherein the operations comprise:
processing the abundance of the at least one food source or the at least one microorganism through a machine learning algorithm to determine a potential hydrogen (pH) value of an oral cavity of the individual, wherein the indicator of an oral or gum disease in the individual is determined based on the pH value of the oral cavity of the individual, and wherein the recommendation for the individual regarding oral health is determined based on the pH value of the oral cavity of the individual.

14. The system of claim 1, wherein the oral biological sample comprises a saliva sample, a biofilm sample, a dental tissue sample, a dental plaque sample, a tartar sample, a dental calculus sample, or any combination thereof.

15. The system of claim 1, wherein the oral biological sample is collected from: a supragingival tissue, a subgingival tissue, a tongue, a buccal mucosa, a soft palate, a hard palate, a floor of a mouth, or any other anatomical location of the oral cavity of the individual.

16. The system of claim 1, wherein the machine learning algorithm comprises a random forest model, a t-distributed stochastic neighbor embedding (tSNE) model, an artificial neural network model, a decision tree model, a k-nearest neighbor (kNN) model, a principal component analysis (PCA) model, a transfer component analysis (TCA) classifier, a deep neural network model, a support vector machine model, or a linear classification model.

17. The system of claim 1, wherein the at least one food source comprises a plant source, an animal source, or an edible fungal source, and wherein the at least one microorganism comprises a bacterium, a virus, or a fungus.

18. The system of claim 1, wherein the recommendation for the individual regarding oral health comprises prebiotics, probiotics, or other diet components to shift a potential hydrogen (pH) of an oral cavity of the individual.

19. A computer-implemented method for providing a recommendation for an individual regarding oral health based on an analysis of an oral biological sample from the individual, the method comprising:
receiving a plurality of sequence reads of the oral biological sample collected from the individual, wherein each of the sequence reads corresponding to one or more nucleic acid molecules from at least one food source or at least one microorganism;
determining an abundance of the at least one food source or the at least one microorganism by:
taxonomically classifying the sequence reads to identify the at least one food source or the at least one microorganism; and
quantifying the one or more nucleic acid molecules from the identified at least one food source or the at least one microorganism;
processing the abundance of the at least one food source or the at least one microorganism through a machine learning algorithm to determine an indicator of an oral or gum disease in the individual, the machine learning algorithm having been trained using a plurality previously received sequence reads of oral biological samples collected from other individuals; and
providing, to a user interface, the recommendation for the individual regarding oral health based on the indicator of the oral or gum disease in the individual.

20. A computer-implemented method for monitoring a progression of an oral and/or gum disease in an individual in need thereof, comprising, the method comprising:
receiving a first plurality of sequence reads from a first oral biological sample of the individual and a second plurality of sequence reads from a second oral biological sample of the individual, the first plurality of sequence reads and the second plurality of sequence reads corresponding to one or more nucleic acid molecules from at least one food source, wherein the first oral biological sample corresponds to a first collection time point and the second oral biological sample corresponds to a second collection time point;
determining a first calculated abundance and a second calculated abundance of the at least one food source by:
taxonomically classifying the first plurality of sequence reads and the second plurality of sequence reads to identify the at least one food source; and
quantifying the one or more nucleic acid molecules from the identified at least one food source;
processing the first calculated abundance and the second calculated abundance of the at least one food source through a machine learning algorithm to determine the progression of the oral or gum disease in the individual, the machine learning algorithm having been trained using a plurality previously received sequence reads of oral biological samples collected from other individuals; and
providing, to a user interface, an indication regarding the progression of the oral or gum disease in the individual.
